(19) United States
(12) Patent Application Publication
(10) Pub. No.: US 2022/0290262 A1
(43) Pub. Date: Sep. 15, 2022

(54) ANTIBODY-DNA CONJUGATES AND HPV DETECTION AND TREATMENT

(71) Applicant: PSOMAGEN INC., Rockville, MD (US)

(72) Inventors: Zachary Apte, San Francisco, CA (US); Jessica Richman, San Francisco, CA (US); Daniel Almonacid, San Francisco, CA (US); Janyra Espinoza, San Francisco, CA (US); Mario Saavedra, San Francisco, CA (US)

(73) Assignee: PSOMAGEN INC., Rockville, MD (US)

(21) Appl. No.: 17/424,641

(22) PCT Filed: Jan. 24, 2020
(86) PCT No.: PCT/US20/14991

§ 371 Date: Jul. 21, 2021

Related U.S. Application Data
(60) Provisional application No. 62/797,175, filed on Jan. 25, 2019.
(60) Provisional application No. 62/797,167, filed on Jan. 25, 2019.
(60) Provisional application No. 62/797,165, filed on Jan. 25, 2019.
(60) Provisional application No. 62/797,173, filed on Jan. 25, 2019.
(60) Provisional application No. 62/797,171, filed on Jan. 25, 2019.
(60) Provisional application No. 62/799,294, filed on Jan. 31, 2019.

(51) Int. Cl. C12Q 1/70; C07K 14/005; C07K 14/335

(52) U.S. Cl. CPC C12Q 1/708; C07K 14/005; C07K 14/335; A61K 38/00

(57) ABSTRACT

The present disclosure related to a method of detecting a molecule using an antibody-DNA conjugate, and pharmaceutical compositions comprising new polypeptides and use thereof.

### CROSS-REFERENCE TO RELATED APPLICATIONS

This application claims the benefit of U.S. Provisional Patent Application No. 62/797,165 filed Jan. 25, 2019 entitled “Beyond Nucleic Acid Detection: DNA-LISA”; U.S. Provisional Patent Application No. 62/797,167 filed Jan. 25, 2019 entitled “Epitope-Based Approach For A HPV6 Treatment And Detection Method, System, And Therapeutic Compositions”; U.S. Provisional Patent Application No. 62/797,171 filed Jan. 25, 2019 entitled “Epitope-Based Approach For A HPV11 Treatment And Detection Method, System, And Therapeutic Compositions”; U.S. Provisional Patent Application No. 62/797,173 filed Jan. 25, 2019 entitled “Epitope-Based Approach For A HPV16 Treatment And Detection Method, System, And Therapeutic Compositions”; U.S. Provisional Patent Application No. 62/797,175 filed Jan. 25, 2019 entitled “Epitope-Based Approach For A HPV18 Treatment And Detection Method, System, And Therapeutic Compositions”, U.S. Provisional Patent Application No. 62/799,294 filed Jan. 31, 2019 entitled “TMA Explorer”, all of which are incorporated by reference herein in their entirety.

A computer readable text file, entitled “SequenceListing.txt,” created on Apr. 10, 2020 with a file size of 30,495 bytes contains the sequence listing for this application and is hereby incorporated by reference in its entirety.

### BACKGROUND

The quantitative detection of a broad spectrum of molecules in biological samples is essential for diagnosis, monitoring, and personalized treatment of diseases. Currently, a variety of techniques such as ELISA (Enzyme linked immunosorbent assays), immunohistochemistry, flow cytometry, spectrophotometry, gas and mass spectrometry are used for the detection of molecules in a sample. Although these techniques have been developed and perfected to be precise and sensitive, they can be limited regarding their scalability, high cost and their ability to detect multiple targets at the same time.

DNA is a versatile molecule which can be used to store a variety of information, from the sequences that encode proteins, to synthetically defined sequences that encode digital data files. As such, synthetic DNA can be attached to macromolecules of interest, and these conjugated complexes can be followed by traditional DNA manipulation techniques, such as polymerase chain reaction (PCR) amplification, qPCR and sequencing. As an example, Immuno-PCR is a technique in which antibodies that bind specific targets are conjugated to defined DNA oligonucleotide molecules, and the binding of target molecules by the antibody-DNA conjugate interest is then followed by conventional PCR or by qPCR.

NGS platforms have had an explosive development, that have allowed to greatly reduce the cost per sample processed, and also allow to query multiple targets in parallel. Antibody-DNA oligo conjugates and their interaction with target molecules can be followed by NGS sequencing, providing an output of improved sensitivity and versatility to current Immuno-PCR techniques.

Current advancements in Next Generation Sequencing (NGS) have allowed to drastically reduce the cost of DNA sequencing, and have propelled several advances in the genomic and transcriptomic field, allowing to now perform detailed analysis that only in recent years were cost-prohibitive. This has also allowed to make genetic testing available to a wide range of users, crossing the barriers of the research lab to the homes of common people. In comparison, the detection and identification of other non-DNA targets still remains in an underdeveloped stage, performed mostly by highly specialized labs, bound by its costs and low scalability.

Products which are used to treat diseases derived from allergens, and pathogens (as virus, bacteria, etc.), usually take advantage of the classic concept of a vaccine, which has been developed to resemble the natural pathogen. Some compositions include some selected antigenic components typical of a pathogen, for example viral capsid proteins. In other cases, a vaccine mimics a weakly pathogenic component, which was obtained by killing, inactivating or attenuating the original allergen or pathogen.

On the other hand, epitope-based vaccines are intended to trigger an immune response through different proteins from a pathogen or allergen, and are focused on some specific short amino acid sequences, which can be isolated to be rationally modified. The ability to rationally modify the sequence of epitopes aimed by in-silico tools, can result in an increase in the activity of those epitopes, which otherwise would not trigger an optimal immunity response. In this regard, an epitope can be a fragment derived from an antigen, which can be recognized by antibodies or immune cells such as B and T cells. Epitopes from protein antigens can be classified into conformational or linear epitopes. Conformational epitopes, are composed of discontinuous sections of a protein, meanwhile linear epitopes are formed by a continuous sequence of amino acids from an antigen. Thereby, the epitope-based approach towards a vaccine development emerges as a potential prevention method of diseases that affect the immune system, as allergy, or to infection diseases. However, additional to a vaccine, other vectors have been used as delivery methods, as DNA vector that codes the peptide (epitope), uses of nanoparticles, polymer-based methods, etc.

The human papillomavirus (HPV) is the most common sexually transmitted infection in the world and the principal agent of cervical cancer (CC), where it has been found in 99% of cases. Additionally, HPV has been found in 93% of anal cancer, 40% of vaginal cancer, 40% of penile cancer and 51% of vulvar cancer cases. The HPV genome is comprised of three regulatory genes involved in transcription and viral replication (E1, E2, E4), three oncogenes (E5, E6, E7) and two genes which constitute the viral capsid (L1, L2).

More than 40 HPV types can be acquired through direct sexual contact by vaginal, anal, and oral sex. The HPV types 6, 11, 32, 40, 42, 43, 44, 54, 55, 61, 62, 64, 70, 71, 72, 74, 81, 83, 84, 87, 89 and 91 (low-risk) have been described as not associated with cancer, but might be associated to genital warts, while HPV types 16, 18, 31, 33, 35, 39, 45, 51, 52, 56, 58, 59, 66, and 68 (high-risk) are responsible for most HPV cancers. Due to the rapid spread of HPV infection, a preventive treatment is essential before cancer progresses.

Currently, several HPV tests have premarket approval in the USA, being the current standard method for the screening of cervical cancer via the Papanicolaou (Pap) test. Other tests are based on HPV nucleic acid screening, which can identify multiple virus types in a semi-quantitative measure, providing an assessment for the presence of high- or low-risk strains, and also viral load. Sensitivity and specificity of these HPV tests are usually higher than that of the PAP smear test in identifying (pre)cancer. For instance, the digene HC2 HPV DNA Test is an in vitro nucleic acid hybridization assay with signal amplification, which is able to qualitative detect by chemiluminescence 5 low risk and 13 high-risk types of HPV DNA in cervical specimens.

To prevent the HPV infection, FDA has approved three commercially available HPV prevention vaccines: Gardasil® and Gardasil® from Merck, and Cervarix® from GlaxoSmithKline. Gardasil (also called recombinant human papillomavirus quadrivalent vaccine) is a vaccine used to prevent anal, cervical, vulvar and vaginal cancer caused by HPV types 16 and 18 and genital warts caused by HPV types 6 and 11. On the other hand, Gardasil 9 (also called recombinant human papillomavirus nonavalent vaccine) prevents anal, cervical, vulvar, and vaginal cancer caused by HPV types 16, 18, 31, 33, 45, 52 and 58 and genital warts caused by HPV types 6 and 11. Finally, Cervarix (also called recombinant human papillomavirus bivalent vaccine) is a vaccine used to prevent cervical cancer caused by HPV types 16 and 18. Those vaccines have been found to provide partial protection against a few additional HPV high risk types via cross-protection.

Those products usually take advantage of the classic concept of vaccine, which has been developed to resemble the natural pathogen. Some compositions include some selected antigenic components typical of a pathogen, for example viral capsid proteins. In other cases, a vaccine mimics a weakly pathogenic component, which was obtained by killing, inactivating or attenuating the original pathogen.

On the other hand, epitope-based vaccines are intended to trigger an immune response through different proteins from a pathogen, and are focused on some specific short amino acid sequences, which can be isolated to be rationally modified. The ability to rationally modify the sequence of epitopes aimed by in-silico tools, can result in an increase in the activity of those epitopes, which otherwise would not trigger an optimal immunity response. In this regard, an epitope can be a fragment derived from an antigen, which can be recognized by antibodies or immune cells such as B and T cells. Epitopes from protein antigens can be classified into conformational or linear epitopes. Conformational epitopes, are comprised of discontinuous sections of a protein, meanwhile linear epitopes are formed by at least a continuous sequence of amino acids from an antigen. Thereby, the epitope-based approach towards a vaccine development emerges as a potential prevention method of HPV infection.

According to previous studies, Lactobacillus species are less abundant in people with HPV infection or cervical cancer condition. Occurrence of HPV infection appears inversely correlated with the presence of some Lactobacillus sp., specially Lactobacillus iners and Lactobacillus crispatus. On the other hand, there are other bacteria that have been inversely correlated with the occurrence of HPV, that is Fusobacterium nucleatum.

### BRIEF SUMMARY OF THE DISCLOSURE

In one aspect, the present disclosure relates to a technology that allows the multiplexable detection of non-DNA compounds and molecules from samples of interest by NGS sequencing, by the use of oligonucleotide-tagged binding proteins. The technology can be adapted to a wide-range of targets, and can be applied to vastly expand the analysis and detection of proteins, lipids, sugars and other target molecules.

In another aspect, the present disclosure relates to a method of generating a vaccine using bacterial-derived (and/or other suitable microorganism-derived) products, proteins and/or epitopes. The bacterial-derived (and/or other suitable microorganism-derived) products, proteins and/or epitopes may include any bacteria or archaea species and/or other suitable taxa, which associate with (e.g., matching, corresponding to, etc.) those found in virus, allergen, and/or any biological entity that possess proteomic material. In an embodiment, bacterial-derived (and/or other suitable microorganism-derived) epitopes are used to trigger an immune response, as common vaccines do, by mimicking the response enabled by some infection, and/or immune reaction. This provides a method of developing preventive vaccines against the effects of viruses, allergens, similar agents, and/or suitable pathogenic agents. In an embodiment, the bacterial-derived products, up to and/or including the bacterium itself, are used to trigger an immune response, as common vaccines do, by mimicking the response enabled by an infection, allergic response, and/or adverse immune response.

In yet another aspect, this disclosure includes at least methods and/or systems that use bacterial-derived products, proteins and epitopes belonging to Lactobacillus sp. and Fusobacterium nucleatum matching those found in HPV proteomes. In one embodiment, Lactobacillus-derived and Fusobacterium nucleatum-derived epitopes are used to trigger an immune response, as common vaccines do, by mimicking the response enabled by HPV infection. This provides a way to develop preventive vaccines against HPV. In another embodiment, the bacterial-derived products, up to and including the bacterium itself, are used to trigger an immune response, as common vaccines do, by mimicking the response enabled by HPV infection.

In some embodiments, the disclosure provides a method for detecting a target molecule, the method comprising the steps of: a) Conjugating of a DNA oligonucleotide to an antibody to the target molecule to form an antibody-DNA oligonucleotide conjugate; b) immobilizing the target molecule; c) binding the target molecule with the antibody-DNA oligonucleotide conjugate; and d) amplifying and detecting the DNA sequence in the antibody-DNA oligonucleotide conjugate.

In some embodiments, the DNA oligonucleotide can comprise any one of the following structures:

5′ R-SPACER-UPST. ADAPTER-DEFINED IDENTIFIER SEQUENCE-DWNST. ADAPTER-3′;

5′ R-SPACER-UPST. ADAPTER-DEFINED IDENTIFIER SEQUENCE-BRIDGE LEFT 3′; or

5′ BRIDGE_RIGHT-DEFINED IDENTIFIER SEQUENCE-DWNST. ADAPTER-SPACER-R3′.

In further embodiments, R is a reactive group, e.g., a group selected from the group consisting of thiol, azide, NETS-ester, amine, aldehyde, hydrazine, hexynyl, octadiynyl dU, acrydite, and sulphydryl.

In still further embodiments, a cross-linker reagent is used in the conjugation step, e.g., SMCC or sulfo-SMCC.

In some embodiments, a reducing agent is used in the conjugation step, e.g., DTT or β-mercaptoethanol.

In some embodiments, the amplification is polymerase chain reaction (PCR) amplification.

In other embodiments, the disclosure provides a polypeptide comprising a sequence, or a sequence having at least 80% identity, e.g., at least 85%, 90%, 95%, 99% identity, to a sequence selected from the group consisting of IGAAIGYFY, GTAGILELL, LVLTLLLYL, SVLVLTLLL, DPYKNLSFW, CAFIVGVLG, RTGISNAST, DSNVRLVVQ, VVLPDPNKF, ISFLGGTVI, VQIAAGTTS, HCYEQLVDS, KHAIVTVTY, KAKQMGLSH, KNALTTAEI, MEAIAKRLD, NTMDYVVWT, TSSETTTPA, VARTLATLL, PNNGKYVMA, NVVKIPPTI, APTITSHPI, PAVSKASAA, NNGKYVMAA, YPDYLQMAA, PVQIAAGTT, KQDILDVLI, TVETTTSSL, PVFITGSGF, HPYFSIKRA, TVQDLKRKY, MESANASTS, PTQHPVTNI, NSHLATPCV, TVARTLATL, IPPTIRHKL, LLLTTPLQF, VLGLLLMHY, QIAAGTTST, RKHKALTLI, VVCFVSIIL, TVVPKVSGY, NGKYVMAAQ, and SRARRRKRA.

In some embodiments, the disclosure provides a polypeptide comprising a sequence, or a sequence having at least 80% identity, e.g., at least 85%, 90%, 95%, 99% identity, to a sequence of X₁X₂X₃X₄X₅X₆X₇X₈X₉, wherein:

X₁ is I, A, E, H, or V;

X₂ is G or Y;

X₃ is A or S;

X₄ is R, Y, A, L, F, H, or P;

X₅ is I, N, D, A, T, or Y;

X₆ is H or O;

X₇ is Y;

X₈ is F or P;

X₉ is Y.

In some embodiments, the disclosure provides a polypeptide comprising a sequence, or a sequence having at least 80% identity, e.g., at least 85%, 90%, 95%, 99% identity, to a sequence of X₁X₂X₃X₄X₅X₆X₇X₈X₉, wherein:

X₁ is P, W or F;

X₂ is V, F, D, or W;

X₃ is F;

X₄ is I;

X₅ is T or P;

X₆ is G, P, A, or C;

X₇ is S, W, Q, F, or P;

X₈ is D or W;

X₉ is F or W.

In some embodiments, the disclosure provides a method for treating or preventing HPV6 infection comprising administering to a patient in need thereof a pharmaceutical composition comprising the polypeptide comprising a sequence, or a sequence having at least 80% identity, e.g., at least 85%, 90%, 95%, 99% identity, to a sequence selected from the group consisting of IGAAIGYFY, GTAGILELL, LVLTLLLYL, SVLVLTLLL, DPYKNLSFW, CAFIVGVLG, RTGISNAST, DSNVRLVVQ, VVLPDPNKF, ISFLGGTVI, VQIAAGTTS, HCYEQLVDS, KHAIVTVTY, KAKQMGLSH, KNALTTAEI, MEAIAKRLD, NTMDYVVWT, TSSETTTPA, VARTLATLL, PNNGKYVMA, NVVKIPPTI, APTITSHPI, PAVSKASAA, NNGKYVMAA, YPDYLQMAA, PVQIAAGTT, KQDILDVLI, TVETTTSSL, PVFITGSGF, HPYFSIKRA, TVQDLKRKY, MESANASTS, PTQHPVTNI, NSHLATPCV, TVARTLATL, IPPTIRHKL, LLLTTPLQF, VLGLLLMHY, QIAAGTTST, RKHKALTLI, VVCFVSIIL, TVVPKVSGY, NGKYVMAAQ, and SRARRRKRA.

In some embodiments, the disclosure provides a method for treating or preventing HPV6 infection comprising administering to a patient in need thereof a pharmaceutical composition comprising the polypeptide comprising a sequence, or a sequence having at least 80% identity, e.g., at least 85%, 90%, 95%, 99% identity, to a sequence of X₁X₂X₃X₄X₅X₆X₇X₈X₉, wherein:

X₁ is I, A, E, H, or V;

X₂ is G or Y;

X₃ is A or S;

X₄ is R, Y, A, L, F, H, or P;

X₅ is I, N, D, A, T, or Y;

X₆ is H or O;

X₇ is Y;

X₈ is F or P;

X₉ is Y.

In some embodiments, the disclosure provides a method for treating or preventing HPV6 infection comprising administering to a patient in need thereof a pharmaceutical composition comprising the polypeptide comprising a sequence, or a sequence having at least 80% identity, e.g., at least 85%, 90%, 95%, 99% identity, to a sequence of X₁X₂X₃X₄X₅X₆X₇X₈X₉, wherein:

X₁ is P, W or F;

X₂ is V, F, D, or W;

X₃ is F;

X₄ is I;

X₅ is T or P;

X₆ is G, P, A, or C;

X₇ is S, W, Q, F, or P;

X₈ is D or W;

X₉ is F or W.

In some embodiments, the disclosure provides a polypeptide comprising a sequence, or a sequence having at least 80% identity, e.g., at least 85%, 90%, 95%, 99% identity, to a sequence selected from the group consisting of PVFITGSDF, LSTPQRLVT, FVVAVLGLL, TPFSPVTPA, LPVVIAFAV, LVAAENDTF, PSDSTVYVP, GAPEVVPPT, SDSTVYVPP, QGNTVEVKF, LVLTLLLYL, RVGLYSRAL, LILPVVIAF, TSSESTTPA, DSNVRLVVE, VLIILISDF, KPRARRRKR, VQIAAATTT, RRLFETREL, LTDAKVALL, MADDSALYE, EVVPVQIAA, NAVYELSDA, SSESTTPAI, QIAAATTTT, KIQSGVRAL, TVARTLGTL, STSATSIDQ, TSSLTITTS, ETNEDILKV, TVQSTTSSL, RALQQVQVT, LKDIVLDLQ, PVQIAAATT, YSIKKVNKT, PRARRRKRA, ASTSATSID, RKHRALTLI, ADDSALYEK, KCKDIRSTL, STTSSLTIT, RKTACRRRL, VVIAFAVCI, AIAKRLDAC, and MEVVPVQIA.

In some embodiments, the disclosure provides a polypeptide comprising a sequence, or a sequence having at least 80% identity, e.g., at least 85%, 90%, 95%, 99% identity, to a sequence of X₁₀X₁₁X₁₂X₁₃X₁₄X₁₅X₁₆X₁₇X₁₈, wherein:

X₁₀ is P, W, or F;

X₁₁ is V, F, D, or W;

X₁₂ is F;

X₁₃ is I;

X₁₄ is T or P;

X₁₅ is G, P, A, or C;

X₁₆ is S, W, Q, F, or P;

X₁₇ is D or W;

X₁₈ is F or W.

In some embodiments, the disclosure provides a method for treating or preventing HPV11 infection comprising administering to a patient in need thereof a pharmaceutical composition comprising the polypeptide comprising a sequence, or a sequence having at least 80% identity, e.g., at least 85%, 90%, 95%, 99% identity, to a sequence selected from the group consisting of PVFITGSDF, LSTPQRLVT, FVVAVLGLL, TPFSPVTPA, LPVVIAFAV, LVAAENDTF, PSDSTVYVP, GAPEVVPPT, SDSTVYVPP, QGNTVEVKF, LVLTLLLYL, RVGLYSRAL, LILPVVIAF, TSSESTTPA, DSNVRLVVE, VLIILISDF, KPRARRRKR, VQIAAATTT, RRLFETREL, LTDAKVALL, MADDSALYE, EVVPVQIAA, NAVYELSDA, SSESTTPAI, QIAAATTTT, KIQSGVRAL, TVARTLGTL, STSATSIDQ, TSSLTITTS, ETNEDILKV, TVQSTTSSL, RALQQVQVT, LKDIVLDLQ, PVQIAAATT, YSIKKVNKT, PRARRRKRA, ASTSATSID, RKHRALTLI, ADDSALYEK, KCKDIRSTL, STTSSLTIT, RKTACRRRL, VVIAFAVCI, AIAKRLDAC, and MEVVPVQIA.

In some embodiments, the disclosure provides a method for treating or preventing HPV11 infection comprising administering to a patient in need thereof a pharmaceutical composition comprising the polypeptide comprising a sequence, or a sequence having at least 80% identity, e.g., at least 85%, 90%, 95%, 99% identity, to a sequence of X₁₀X₁₁X₁₂X₁₃X₁₄X₁₅X₁₆X₁₇X₁₈, wherein:

X₁₀ is P, W, or F;

X₁₁ is V, F, D, or W;

X₁₂ is F;

X₁₃ is I;

X₁₄ is T or P;

X₁₅ is G, P, A, or C;

X₁₆ is S, W, Q, F, or P;

X₁₇ is D or W;

X₁₈ is F or W.

In some embodiments, the disclosure provides a polypeptide comprising a sequence, or a sequence having at least 80% identity, e.g., at least 85%, 90%, 95%, 99% identity, to a sequence selected from the group consisting of LLKLVGSTS, LLTLLGSPW, LLMLLGLTW, LGKWLGSTW, LAKLLGSGW, SAFLKSNSQ, PPTPAETGG, VEKKTGDAI, FWLQPLADA, QPPTPAETG, SGKSIGAKV, PYLHNRLVV, AHTKDGLTV, VAVNPGDCP, SHAASPTSI, TPAILDINN, AEEIELQTI, NALDGNLVS, FELSQMVQW, IAEQILQYG, GGLGIGTGS, TAHALFTAQ, VPTLAVSKN, ADPAAATKY, YDLSTIDPA, AGTSRLLAV, NASAFLKSN, TLCQRLNVC, HAASPTSIN, YSLYGTTLE, LGKRKATPT, CEEASVTVV, LWLPSEATV, IPIVPGSPQ, MADPAAATK, KPYWLQRAQ, DPAGTNGEE, LAKFKELYG, IGNKQTLRT, GNQLFVTVV, DAGDFYLHP, YGNTEVETQ, TPPRPIPKP, AAMLAKFKE, PFDENGNPV, LGIGTGSGT, ATKYPLLKL, GEDLVDFIV, INHQVVPTL, TPSIADSIK, ICEEASVTV, LYLHIQSLA, LADTNSNAS, DYLTQAETE, SLIPIVPGS, and RAAKRRLFE.

In some embodiments, the disclosure provides a polypeptide comprising a sequence, or a sequence having at least 80% identity, e.g., at least 85%, 90%, 95%, 99% identity, to a sequence of X₁₉X₂₀X₂₁X₂₂X₂₃X₂₄X₂₅X₂₆X₂₇, wherein:

X₁₉ is F, W, Y, A, or I;

X₂₀ is W, Y, F, G, I, L, A, C, S, R, T, Q, or V;

X₂₁ is L, N, D, W, F, P, Q, V, I, E, R, A, C, T, Y, H, or S;

X₂₂ is Q, D, P, N, S, A, E, L, F, C, W, H, I, T, G, or K;

X₂₃ is P, D, or H;

X₂₄ is L, E, N, F, Y, G, P, T, D, A, Q, I, V, or H;

X₂₅ is A, W, F, Y, E, Q, V, G, L, P, or M;

X₂₆ is D, Y, T, A, W, P, F, S, Q, E, K, R, H, V, or C;

X₂₇ is A, F, L, W, P, H, V, S, N, C, E, or M.

In some embodiments, the disclosure provides a method for treating or preventing HPV16 infection comprising administering to a patient in need thereof a pharmaceutical composition comprising a polypeptide comprising a sequence selected from the group consisting of LLKLVGSTS, LLTLLGSPW, LLMLLGLTW, LGKWLGSTW, LAKLLGSGW, SAFLKSNSQ, PPTPAETGG, VEKKTGDAI, FWLQPLADA, QPPTPAETG, SGKSIGAKV, PYLHNRLVV, AHTKDGLTV, VAVNPGDCP, SHAASPTSI, TPAILDINN, AEEIELQTI, NALDGNLVS, FELSQMVQW, IAEQILQYG, GGLGIGTGS, TAHALFTAQ, VPTLAVSKN, ADPAAATKY, YDLSTIDPA, AGTSRLLAV, NASAFLKSN, TLCQRLNVC, HAASPTSIN, YSLYGTTLE, LGKRKATPT, CEEASVTVV, LWLPSEATV, IPIVPGSPQ, MADPAAATK, KPYWLQRAQ, DPAGTNGEE, LAKFKELYG, IGNKQTLRT, GNQLFVTVV, DAGDFYLHP, YGNTEVETQ, TPPRPIPKP, AAMLAKFKE, PFDENGNPV, LGIGTGSGT, ATKYPLLKL, GEDLVDFIV, INHQVVPTL, TPSIADSIK, ICEEASVTV, LYLHIQSLA, LADTNSNAS, DYLTQAETE, SLIPIVPGS, and RAAKRRLFE.

In some embodiments, the disclosure provides a method for treating or preventing HPV16 infection comprising administering to a patient in need thereof a pharmaceutical composition comprising a polypeptide or a polypeptide comprising at least 80% identity, e.g., at least 85%, 90%, 95%, 99% identity, to a sequence of X₁₉X₂₀X₂₁X₂₂X₂₃X₂₄X₂₅X₂₆X₂₇, wherein:

X₁₉ is F, W, Y, A, or I;

X₂₀ is W, Y, F, G, I, L, A, C, S, R, T, Q, or V;

X₂₁ is L, N, D, W, F, P, Q, V, I, E, R, A, C, T, Y, H, or S;

X₂₂ is Q, D, P, N, S, A, E, L, F, C, W, H, I, T, G, or K;

X₂₃ is P, D, or H;

X₂₄ is L, E, N, F, Y, G, P, T, D, A, Q, I, V, or H;

X₂₅ is A, W, F, Y, E, Q, V, G, L, P, or M;

X₂₆ is D, Y, T, A, W, P, F, S, Q, E, K, R, H, V, or C;

X₂₇ is A, F, L, W, P, H, V, S, N, C, E, or M.

In some embodiments, the disclosure provides a polypeptide comprising a sequence, or a sequence having at least 80% identity, e.g., at least 85%, 90%, 95%, 99% identity, to a sequence selected from the group consisting of QKQLEILGC, GGQTVQVYF, QATTKDGNS, KNGNPVYEI, HRFSTSDDT, KGGQTVQVY, KSRLTVAKG, ICGHYIILF, QHRFSTSDD, KQGAMLAVF, KAHKAIELQ, SIVDLSTHF, and ETLSERLSC.

In some embodiments, the disclosure provides a polypeptide comprising a sequence, or a sequence having at least 80% identity, e.g., at least 85%, 90%, 95%, 99% identity, to the sequence of X₂₈X₂₉X₃₀X₃₁X₃₂X₃₃X₃₄X₃₅X₃₆, wherein:

X₂₈ is G, W, F, Y, P, R, C, I or L;

X₂₉ is G, P, D, F, A, Q, Y or S;

X₃₀ is Q, W, Y, H, R, V, F, L, P, A, D, G or S;

X₃₁ is T, W, A, F, P, G, H, R, Y, D, N, Q or S;

X₃₂ is V, W, G or T;

X₃₃ is Q, N, E, G, P or W;

X₃₄ is V, D, T or A;

X₃₅ is Y, D, W or F;

X₃₆ is F or W.

In some embodiments, the disclosure provides a method for treating or preventing HPV18 infection comprising administering to a patient in need thereof a pharmaceutical composition comprising a polypeptide comprising a sequence, or a sequence having at least 80% identity, e.g., at least 85%, 90%, 95%, 99% identity, to a sequence selected from the group consisting of QKQLEILGC, GGQTVQVYF, QATTKDGNS, KNGNPVYEI, HRFSTSDDT, KGGQTVQVY, KSRLTVAKG, ICGHYIILF, QHRFSTSDD, KQGAMLAVF, KAHKAIELQ, SIVDLSTHF, and ETLSERLSC.

In some embodiments, the disclosure provides a method for treating or preventing HPV18 infection comprising administering to a patient in need thereof a pharmaceutical composition comprising a polypeptide comprising a sequence, or a sequence having at least 80% identity, e.g., at least 85%, 90%, 95%, 99% identity, to a sequence of X₂₈X₂₉X₃₀X₃₁X₃₂X₃₃X₃₄X₃₅X₃₆, wherein:

X₂₈ is G, W, F, Y, P, R, C, I or L;

X₂₉ is G, P, D, F, A, Q, Y or S;

X₃₀ is Q, W, Y, H, R, V, F, L, P, A, D, G or S;

X₃₁ is T, W, A, F, P, G, H, R, Y, D, N, Q or S;

X₃₂ is V, W, G or T;

X₃₃ is Q, N, E, G, P or W;

X₃₄ is V, D, T or A;

X₃₅ is Y, D, W or F;

X₃₆ is F or W.

### BRIEF DESCRIPTION OF THE DRAWINGS

FIG. 1 illustrates a method for NGS detection of molecules with DNA oligo-tagged proteins.

FIG. 2 illustrates a scheme of multiplexing detection of different targets by oligo/antibodies coupled with NGS.

FIG. 3 illustrates a scheme of molecule interaction detection by oligonucleotide-tagged binding proteins coupled with NGS.

FIG. 4 illustrate a workflow of evaluation of host-microbiome interactions.

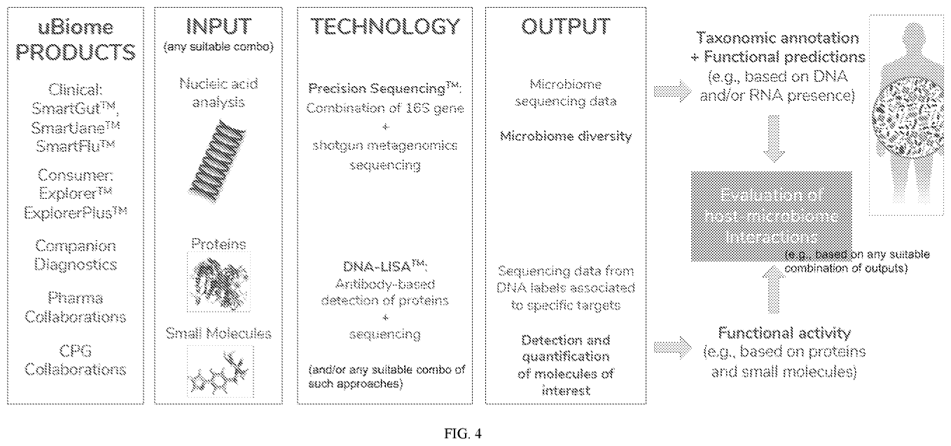

FIG. 5 is a schematic diagram of DNA-labelled antibodies used for target identification.

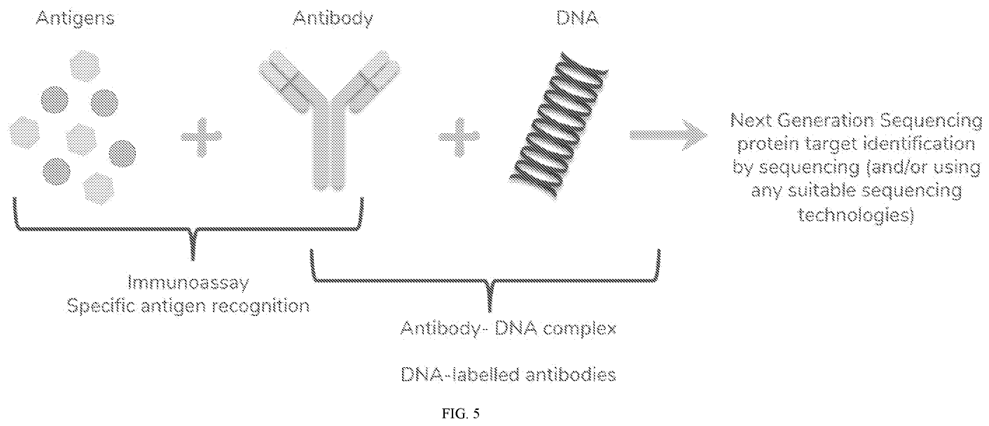

FIG. 6 illustrates a schematic diagram of using DNA-labelled antibodies in direct DNA-LISA and Sandwich DNA-LISA.

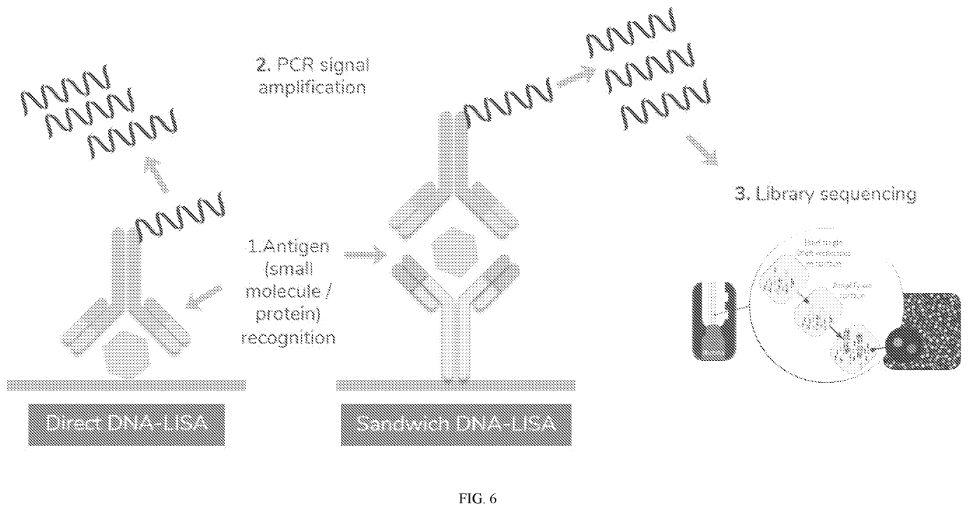

FIG. 7 illustrates a general view of a workflow employed to identify “de novo” epitopes from pathogen proteins associated to a particular condition.

### DETAILED DESCRIPTION OF THE DISCLOSURE

Current molecule detection platforms can detect a limited amount of targets at the same time, and can have problems regarding price and scalability. The technology disclosed herein allows to couple NGS with the detection of a variety of non-DNA molecules, increasing the versatility of this platform, and providing all the benefits of NGS detection for the detection of other type of non-DNA compounds. The technology can be applied to develop platforms that will greatly reduce the cost, improve the versatility, scalability and multiplexing for detection of target molecules.

Embodiments of a method for NGS detection of molecules with DNA oligo-tagged proteins comprises the steps of:
1) Conjugation of DNA oligonucleotides to binding proteins to form protein-DNA oligonucleotide conjugate;
2) Target immobilization;
3) Target binding with protein-DNA oligonucleotide conjugate; and
4) Signal amplification and sequencing detection.
   Each of these steps is described in more detail below.

### Step 1. Conjugation of DNA Oligonucleotides to Binding Proteins

First, DNA oligonucleotides have to be conjugated to binding proteins of interest, which can be any protein that specifically and avidly binds a target molecule, including but not restricted to antibodies, carbohydrate-binding proteins, lipid-binding proteins, DNA-binding proteins, small-molecule binding proteins, lectins, LPS-binding proteins, transcription factors, metal-binding proteins, vitamin-binding proteins, CRISPR proteins, TALEN proteins and enzymatically inactive proteins. In some embodiments, the DNA oligonucleotides used have the following structure:

5′ R-SPACER-UPST. ADAPTER-DEFINED IDENTIFIER SEQUENCE-DWNST. ADAPTER-3′

The R group serves as the reactive group used to conjugate the DNA oligonucleotide to the binding protein of interest. This reactive group can be a thiol, azide or NHS-ester, amine, aldehyde, hydrazine, hexynyl, octadiynyl dU, acrydite, sulphydryl or any other group susceptible to be reduced. SPACER region corresponds to any stretch of nucleotides that physically separates the tethering R group from the rest of the DNA oligonucleotide. UPST. ADAPTER and DWNST. ADAPTER correspond to the upstream and downstream adapter sequences respectively, that will be used amplify the oligonucleotide signal in downstream processes, and which may or may not already contain sequences to allow identification by the NGS platform of interest. DEFINED IDENTIFIER SEQUENCE corresponds to defined unique DNA sequence that will allow to track the tagged protein after sequencing is performed.

In a first variation, other types of nucleic acids that contain the same sequence elements can be conjugated to the binding protein instead of a single stranded DNA oligonucleotide, including double stranded DNA and RNA. In the latter case, additional steps have to be performed for signal amplification in STEP 4.

The protein-DNA conjugation proceeds through the mixing of the binding protein(s) of interest with a cross-linker reagent (e.g. succinimidyl 4-(N-maleimidomethyl)cyclohexane-1-carboxylate (SMCC) or sulfosuccinimidyl 4-(N-maleimidomethyl)cyclohexane-1-carboxylate (sulfo-SMCC) and reduced R oligonucleotides through incubation with a reducing agent (e.g. DTT, β-mercaptoethanol, or other suitable reducing agent). After the incubation period, the excess of cross-linker reagent and reducing agent are removed using desalting columns. Then, binding proteins and R oligos are mixed and dialysed for the buffer exchange and subsequent conjugation to occur. This protocol allows to prepare in parallel, multiple binding protein-DNA conjugates, with a different DEFINED IDENTIFIER SEQUENCE tagged to each different binding protein-DNA conjugate for the downstream NGS based differentiation of multiple targets.

The binding protein-DNA conjugates are then purified to remove free DNA oligonucleotides by affinity purification, by the use of protein a resin and/or protein G resin; or by size exclusion purification with chromatographic resins or centrifugation-based filters. Alternatively, free, unconjugated DNA oligos can be removed with a 5′ exonuclease enzyme, that does not target 5′ tethered DNA for degradation. At the end of this step, protein-DNA conjugates are obtained, for their use in the following steps.

### Step 2. Target Immobilization

In order to detect the desired target molecule(s) from a sample of interest, the components of the sample are first immobilized in a solid surface, including but not limited to multiple well plates, PCR-tubes, magnetic beads, or binding resins. For the immobilization in material containers (e.g. polycarbonate, polypropylene or polystyrene), the sample of interest, being this sample any type of solution, from human, animal, environmental or synthetic origin, is mixed with coating buffer (e.g. sodium bicarbonate alkaline pH) added to the container, and incubated at a temperature between about 4° C.-10° C. Alternatively, coating buffer can be supplemented with a fixing reagent (e.g. glutaraldehyde) in order to improve sample binding to the container. After the incubation, the liquid volume is removed and the container rinsed three times with washing solution (e.g. Tween 20 in PBS, or other diluted solution of a non-ionic detergent in a saline buffer). After removing washing solution, the container with bound sample solution is treated with blocking solution (e.g. BSA in PBS, or other defined protein-rich solution, like milk solution in PBS). After incubation, and removal of blocking solution, the container is rinsed with washing solution. After this step, the immobilized sample is ready to be incubated with the binding protein-DNA conjugates.

In some embodiments, a directed capture immobilization of target molecules can be performed, by first immobilizing an antibody or other specific binding protein with the protocol described above, to a container. Then, the sample of interest is added to the container and the molecules to be detected are affinity-captured by the immobilized protein.

Step 3. Target Binding with Protein-DNA Oligonucleotide Conjugate

Single or multiple binding protein-DNA conjugates are added to the container that has the immobilized sample, in an amount that can be optimized for each specific binding protein-DNA conjugate and each specific target, and that can go from direct addition to dilutions of the protein-DNA conjugate, prepared in the blocking solution described above. After incubation at room temperature, binding protein-DNA conjugate solution is removed, and the container is rinsed repeatedly with washing solution. After this step, the binding protein-DNA conjugates are specifically bound to the target of interest in the immobilized sample, and the protocol can proceed to the signal amplification step.

In some embodiments, if the working sample contains a molecule that is ubiquitous to this type of sample (e.g., actin or GAPDH proteins in animal samples), a binding protein-DNA conjugate targeting this type of molecule can be also added in conjunction with other conjugates. This will allow the detection of the ubiquitous molecule (which will vary in quantity depending on the amount of sample processed), and allow to normalize the signal (sequencing reads) of the target molecule with the signal of the ubiquitous molecule, to help to compare the data obtained across different samples. If no known ubiquitous molecule is present in the working sample, a defined-concentration of a known molecule can be added during the immobilization step as, and this internal control molecule also targeted by a binding protein-DNA conjugate, and its signal used to normalize the signal of other targeted molecules of unknown concentration, and to estimate absolute amounts of the target molecule.

In some embodiments, a proximity assay variation can be performed during target binding, wherein two individual binding proteins are tagged as detailed in STEP 1 with one of the following oligonucleotides

OligoA: 5′ R-SPACER-UPST. ADAPTER-DEFINED IDENTIFIER SEQUENCE-BRIDGE_LEFT 3′; or

OligoB: 5′ BRIDGE_RIGHT-DEFINED IDENTIFIER SEQUENCE-DWNST. ADAPTER-SPACER-R3′

These tagged binding proteins are added to the immobilized sample, and then a bridging oligonucleotide is added, that is complementary to the BRIDGE_LEFT and BRIDGE_RIGHT sections of each oligonucleotide. If the two individual tagged binding proteins are in proximity in the sample, the bridging oligonucleotide will bind by DNA complementarity both oligonucleotides bound to binding proteins. After this, any DNA ligating enzyme can be added, that will ligate the 3′ end of Oligo1 to the 5′ end of Oligo2, thus generating a linear molecule that can be amplified and detected as explained in STEP 4. If this proximity assay is performed using two individual binding proteins that target different sections of a same molecule, it can be used to improve sensitivity and precision of the technique. If this proximity assay is performed using two individual binding proteins that target different molecules, this approach can be used to query the proximity and interaction of the two molecules in the space of the immobilized sample.

### Step 4. Signal Amplification and Sequencing Detection

A PCR amplification is performed in the container with the immobilized sample and attached binding protein-oligo conjugate, using the following DNA oligonucleotide primers

5′-SEQUENCING ADAPTER-SEQUENCING INDEX-UPST. ADAPTER-3′

5′-SEQUENCING ADAPTER-SEQUENCING INDEX-DWNST. ADAPTER-3′

The reaction proceeds using at least between 0.01-0.10 units/uL of DNA polymerase, and between 5-45 cycles of PCR. This allows the amplification of the oligonucleotide sequences that are conjugated to the targeting proteins of interest, and adds as well sequencing adapter and index sequences required for NGS.

Additionally to the normalizing steps described in the target binding step, defined concentration of DNA molecules that are amplified with the oligonucleotide primers described above can be added as an internal control to the signal amplification reaction, in order to correlate the signal (sequencing reads) obtained for a defined concentration internal control with the sequencing reads of target molecules of unknown concentration, in order to estimate an absolute value of the amount of target molecule.

In some embodiments, this method can optionally include a second step of PCR amplification, that uses primers that anneal at the sequencing adapters added by the second PCR. This step can be repeatedly used when product concentrations from the two-step PCR are in low concentrations that are not sufficient for NGS.

After all steps of amplification are finished, obtained PCR product libraries are suitable for NGS. After DNA sequencing is performed, reads are mapped to a database containing the DEFINED IDENTIFIER SEQUENCES of the protein-DNA conjugates used, in order to detect target molecules of interest. Mapped reads numbers can be normalized with the reads obtained for the ubiquitous target molecules in a sample (as explained in STEP 3) in order to normalize the signal of target molecules to the amount total sample. Also, mapped reads from target molecules can be compared to mapped reads obtained from defined concentration molecule or DNA internal controls (As explained in STEP 3 and STEP 4 respectively) in order to estimate the amount of target molecules in the sample of interest.

In some embodiments, the present disclosure relates to a method to conjugate specific DNA oligonucleotides to molecule-binding proteins.

In some embodiments, the present disclosure relates to a method to non-specifically immobilize target molecules in a sample to a container.

In some embodiments, the present disclosure relates to a method to specifically capture target molecules in a sample to a container.

In some embodiments, the present disclosure relates to a method to detect target molecules from samples of interest using molecule-targeting protein-DNA conjugates by NGS.

In some embodiments, the present disclosure relates to a method to use two oligonucleotide-tagged binding proteins targeted to the same molecule to improve sensitivity and specificity of detection by NGS.

In some embodiments, the present disclosure relates to a method to use two oligonucleotide-tagged binding proteins targeted to different molecules (for example: B amiloide monomers, DNA binding protein) to query proximity and interaction of those molecules by NGS.

In some embodiments, the present disclosure relates to a method to use two oligonucleotide-tagged binding proteins targeted to different molecules to query proximity and interaction of those molecules by NGS, as diagnosis of an specific health condition (e.g autoimmune condition), with or without combination of other sequences detection targets techniques (e.g. FISH, Microarrays and/or other transcriptomics techniques).

In some embodiments, the present disclosure relates to a method for treatment of a specific health condition, where use two oligonucleotide-tagged binding proteins targeted to different molecules to query proximity and interaction of those molecules by NGS, sequence information can be used as labelling technique for further site-directed treatment.

In some embodiments, the present disclosure relates to a method for identification of specific molecules, by using two oligonucleotide-tagged binding proteins targeted to same and/or different molecules to query proximity and interaction of those molecules by NGS.

In some embodiments, the present disclosure relates to a method to normalize the signal of molecules of interest detected using molecule-targeting protein-DNA conjugates by NGS, by detecting also molecules of ubiquitous nature in the samples of interest.

In some embodiments, the present disclosure relates to a method to determine absolute quantities of molecules of interest detected using molecule-targeting protein-DNA conjugates by NGS, by comparing sequencing reads to those obtained for DNA and non-DNA internal control molecules.

### P60-PRV2

In some embodiments, the present disclosure relates to a powerful method of immuno-detection which combines the specificity of an ELISA with the sensitivity of PCR amplification and sequencing identification.

In some embodiments, the present disclosure relates to identification of several targets at the same time via multiplex reaction paired with sequencing allowing the identification of a broad panel of biological markers.

In some embodiments, the present disclosure relates to a flexible detection platform, adaptable to any protein and small molecules for which there are antibodies thereof available.

In some embodiments, the present disclosure relates to a flexible detection platform that can be used for as diagnostic and monitoring tool for inflammatory disorders; for example, IBD diagnostic markers for SG (calprotectin and lactoferrin); intestinal permeability integrity evaluation for SG and Explorer (leaky gut syndrome: zonulin, fecal fat); food allergies detection for SG and Explorer (secretory IgA); antiviral response measurement for SF (interferon response associated molecules).

In some embodiments, the present disclosure relates to a flexible detection platform that can be used for as diagnostic and monitoring tool for cancer, for example, colorectal cancer (CRC) markers detection for SG or for a CRC panel (CEA, TIMP-1, occult blood), and gastric cancer biomarkers.

In some embodiments, the present disclosure relates to a flexible detection platform that can be used for as diagnostic and monitoring tool for autoimmune diseases, for example, serum sample analysis for autoantibodies (e.g. celiac, rheumatoid arthritis, type I diabetes and lupus disease panel).

In some embodiments, the present disclosure relates to a flexible detection platform that can be used for as diagnostic and monitoring tool for evaluation of nutritional status, for example, serum sample analysis for vitamins.

In some embodiments, the present disclosure relates to a flexible detection platform that can be used for as diagnostic and monitoring tool for detection of viral, bacterial and parasitic antigens, for example, evaluation of active production of toxin A and B from Clostridium difficile for SG, and antibody response test against pathogens in vaginal swabs for SJ.

In some embodiments, the present disclosure relates to a method to predict de novo epitopes against the pathogen/allergen agent, based on microbiota species present in mouth, gut, vaginal, mouth, skin, genitals, and/or any suitable body sites (e.g., healthy sites) in relation to the microbiome.

A strategy to identify T cell epitopes can include first predicting HLA binding peptides by in-silico methods. That short length peptides (8-20 amino acids), are predicted with the support of methods that include neural networks (ANN), support vector machine (SVM), matrix based (MB) algorithms, and/or their combination, and/or any suitable artificial intelligence approach and/or analytical technique, including any one or more of supervised learning (e.g., using logistic regression, using back propagation neural networks, using random forests, decision trees, etc.), unsupervised learning (e.g., using an Apriori algorithm, using K-means clustering), semi-supervised learning, a deep learning algorithm (e.g., neural networks, a restricted Boltzmann machine, a deep belief network method, a convolutional neural network method, a recurrent neural network method, stacked auto-encoder method, etc.), reinforcement learning (e.g., using a Q-learning algorithm, using temporal difference learning), a regression algorithm (e.g., ordinary least squares, logistic regression, stepwise regression, multivariate adaptive regression splines, locally estimated scatterplot smoothing, etc.), an instance-based method (e.g., k-nearest neighbor, learning vector quantization, self-organizing map, etc.), a regularization method (e.g., ridge regression, least absolute shrinkage and selection operator, elastic net, etc.), a decision tree learning method (e.g., classification and regression tree, iterative dichotomiser 3, C4.5, chi-squared automatic interaction detection, decision stump, random forest, multivariate adaptive regression splines, gradient boosting machines, etc.), a Bayesian method (e.g., naïve Bayes, averaged one-dependence estimators, Bayesian belief network, etc.), a kernel method (e.g., a support vector machine, a radial basis function, a linear discriminate analysis, etc.), a clustering method (e.g., k-means clustering, expectation maximization, etc.), an associated rule learning algorithm (e.g., an Apriori algorithm, an Eclat algorithm, etc.), an artificial neural network model (e.g., a Perceptron method, a back-propagation method, a Hopfield network method, a self-organizing map method, a learning vector quantization method, etc.), a dimensionality reduction method (e.g., principal component analysis, partial least squares regression, Sammon mapping, multidimensional scaling, projection pursuit, etc.), an ensemble method (e.g., boosting, bootstrapped aggregation, AdaBoost, stacked generalization, gradient boosting machine method, random forest method, etc.). Additionally or alternatively, any suitable portions of embodiments of the method described herein can include, apply, employ, perform, use, be based on, and/or otherwise be associated with artificial intelligence approaches and/or analytical techniques described herein.

In some embodiments, the method described herein predicts peptides which bind to human leukocyte antigen (HLA) class I and II alleles, that correspond to the human version of the major histocompatibility complex (MHC). HLA complex can present those peptide antigens as epitopes.

In some embodiments, the method comprises one or more following steps.

First, the microbiota which is present in the healthy that can be related with the disease (e.g., microorganism-related condition) is identified. At the same time, allergens, proteins or agents which can be related with the disease or condition are identified.

Then, a preliminary group of de novo epitopes is obtained through the epitopes prediction methods; this preliminary group of de novo epitopes are filtered, where repetitive sequences of epitopes are removed, and the proteomes of the microbiome, bacteria in healthy patient are analyzed. With those filtered epitopes, a new database is made.

Each epitope from the database is correlated with proteome sequences obtained from an inversely-correlated organism proteome database, through local pairwise alignment tools in order to find “de novo” predicted epitopes in those proteomes. The epitope will be considered as “common epitopes” compared to the predicted epitopes, according the following criteria:

In case of MHC type I:
1. Sequences having more than 70% identity and 100% matches*.
2. Sequences having 100% identity and more or equal than 7 matches*.

In case of MHC is type II:
3. Sequences having more than 60% identity and 100% matches*.
4. Sequences having 100% identity and more or equal than 9 matches*.

Where “match” is the local similarity of an amino acid position in a pairwise alignment.

However, any suitable criteria (e.g., any suitable percent identity, percent matches, number of matches, any suitable percent similarity such as 60% similarity, etc.) can be used.

In variations, common epitope sequences that are part of associated bacteria and/or other suitable types of microorganisms described in literature, e.g, that are directly involved in the triggering of diseases are discarded.

In variations, the common epitopes can be grouped, by agent, identity and/or MHC allele best affinity.

To classify “common epitopes” according their affinity to the receptor, we tested our database of filtered de novo epitopes against a protein receptor class I/II structure using molecular docking simulations, but any suitable simulations and/or processes can additionally or alternatively be performed.

In a next stage, in a variation, to improve the affinity of epitopes for the MHC receptor, it is also possible to subject the best epitopes to a reengineering, which means that every amino acid can be mutated in-silico, one at the time, by the other 21 proteinogenic amino acids (Alanine, Arginine, Asparagine, Aspartic acid, Cysteine, Glutamic acid, Glutamine, Glycine, Histidine, Isoleucine, Leucine, Lysine, Methionine, Phenylalanine, Proline, Serine, Threonine, Tryptophan, Tyrosine, Valine, Selenocysteine and Pyrrolysine). Those new epitopes obtained by reengineering can be tested by docking and/or other suitable techniques, and then classified according their energy of binding to the receptor. In this way, it is possible to obtain new epitopes with a better affinity to the receptor.

In specific examples, a de-novo list of epitopes are then obtained which are final candidates to be used as preventative, treatment, therapeutics and/or diagnostics for one or more diseases (e.g., microorganism-related conditions).

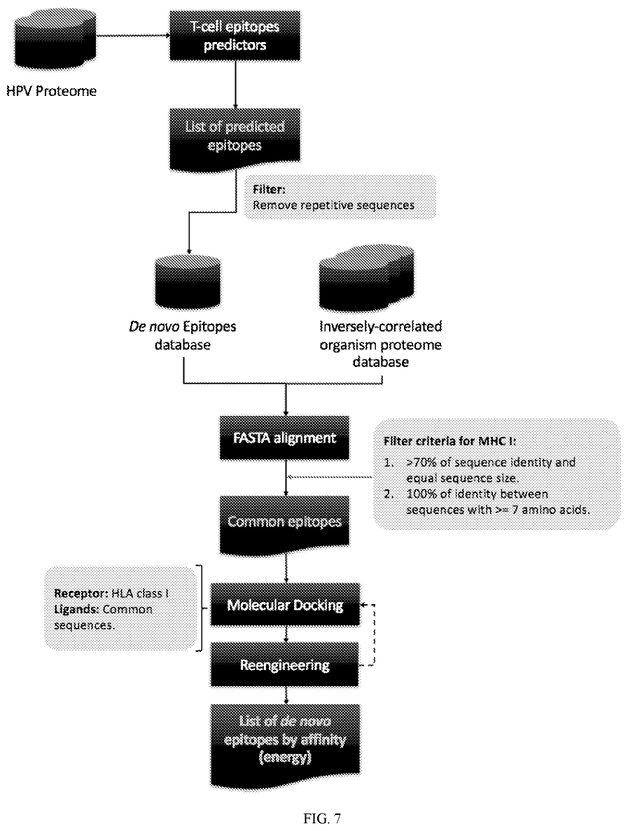

Accordingly, in specific examples, a general view of the workflow employed to identify “de novo” epitopes from pathogen proteins associated to a particular condition, and then search them in proteomes from inversely-associated organisms, can be summarized in FIG. 7.

Additional embodiments of the present disclosure (e.g., of the method, of diagnostics, of therapeutic compositions, etc.) can include, be for, be performed for, apply, correspond to, be diagnostic of (e.g., for diagnosing, etc.), be therapeutic of (e.g., therapeutic composition including epitopes therapeutic of, etc.), and/or otherwise be associated with one or more conditions, including any one or more of: diseases, symptoms, causes (e.g., triggers, etc.), disorders, associated risk (e.g., propensity scores, etc.), associated severity, behaviors (e.g., caffeine consumption, habits, diets, etc.), and/or any other suitable aspects associated with conditions. Conditions can include one or more disease-related conditions, which can include any one or more of: HPV, gastrointestinal-related conditions (e.g., irritable bowel syndrome, inflammatory bowel disease, ulcerative colitis, celiac disease, Crohn's disease, bloating, hemorrhoidal disease, constipation, reflux, bloody stool, diarrhea, etc.); allergy-related conditions (e.g., allergies and/or intolerance associated with wheat, gluten, dairy, soy, peanut, shellfish, tree nut, egg, etc.); skin-related conditions (e.g., acne, dermatomyositis, eczema, rosacea, dry skin, psoriasis, dandruff, photosensitivity, etc.); locomotor-related conditions (e.g., gout, rheumatoid arthritis, osteoarthritis, reactive arthritis, multiple sclerosis, Parkinson's disease, etc.); cancer-related conditions (e.g., lymphoma; leukemia; blastoma; germ cell tumor; carcinoma; sarcoma; breast cancer; prostate cancer; basal cell cancer; skin cancer; colon cancer; lung cancer; cancer conditions associated with any suitable physiological region; etc.), cardiovascular-related conditions (e.g., coronary heart disease, inflammatory heart disease, valvular heart disease, obesity, stroke, etc.), anemia conditions (e.g., thalassemia; sickle cell; pernicious; fanconi; haemolyitic; aplastic; iron deficiency; etc.), neurological-related conditions (e.g., ADHD, ADD, anxiety, Asperger's syndrome, autism, chronic fatigue syndrome, depression, etc.), autoimmune-related conditions (e.g., Sprue, AIDS, Sjogren's, Lupus, etc.), endocrine-related conditions (e.g., obesity, Graves' disease, Hashimoto's thyroiditis, metabolic disease, Type I diabetes, Type II diabetes, etc.), Lyme disease conditions, communication-related conditions, sleep-related conditions, metabolic-related conditions, weight-related conditions, pain-related conditions, genetic-related conditions, chronic disease, and/or any other suitable type of disease-related conditions. Additionally or alternatively, microorganism-related conditions can include one or more human behavior conditions which can include any one or more of: caffeine consumption, alcohol consumption, other food item consumption, dietary supplement consumption, probiotic-related behaviors (e.g., consumption, avoidance, etc.), other dietary behaviors, habitué behaviors (e.g., smoking; exercise conditions such as low, moderate, and/or extreme exercise conditions; etc.), menopause, other biological processes, social behavior, other behaviors, and/or any other suitable human behavior conditions. Conditions can be associated with any suitable phenotypes (e.g., phenotypes measurable for a human, animal, plant, fungi body, etc.).

In some embodiments, the predicted epitopes from any strains of bacteria or archaea species or reengineered ones, and/or other suitable microorganisms, can be used in different products for the diagnostics, treatment and/or prevention and/or suitable conditions described herein, such as based on approaches described herein.

In some embodiments, the therapeutic composition described herein includes one or more epitopes described herein, such as epitopes derived from microorganisms described herein and/or other suitable microorganisms, such as for use in diagnostics, therapeutics, and/or other suitable applications, such as in relation to one or more conditions (e.g. Addison's disease, Alzheimer's disease, Attention Deficit Hyperactivity Disorder, Anemia, Anxiety, Asperger's Syndrome, Asthma, Atrial Fibrillation, Autism, Bronchitis, Cancer, Chronic Fatigue Syndrome, Cirrhosis, Dementia, Depression, Diabetes Type I, Diabetes Type II, Epilepsy, Epstein-Barr Virus I, Fibromyalgia, Glaucoma, Graves disease, Hashimoto's thyroiditis, Hemorrhoids, High blood pressure, High cholesterol, Hypertension, Hypothyroidism, Insomnia, Lupus, Lyme Disease, Migraine, Mitral, Valve Prolapse, Multiple Sclerosis, Obesity, Osteoarthritis, Parkinson's disease, Pneumonia, Rheumatoid Arthritis, Sinusitis, Strep throat, Stroke, Cystic Fibrosis, Acid reflux/GERD, Celiac disease, Crohn's disease, Irritable Bowel Syndrome, Ulcerative Colitis, Bacterial vaginosis, Endometriosis, Painful periods, PolyCystic Ovarian Syndrome, Trichomoniasis, Vulvodynia, Yeast infection, Acne, Eczema, Psoriasis, Rosacea, Dental decay, Ectodermal dysplasia, Gingivitis, Oral herpes, Periodontal disease, Sjogren's syndrome, Chlamydia, Gonorrhea, Herpes, warts, HIV, Human Papillomavirus, Syphilis, Bloody stool, Brain fog, Cold/cough, Constipation, Diarrhea, Dizziness, Fever, Headache, Insomnia, Migraine, Muscle aches, Rash, Stomach pain, or other where virus, bacteria or some external agent is related with) described herein, such as based on approaches described herein. Additionally or alternatively, embodiments (e.g., of the method, of diagnostics, of therapeutic compositions, etc.) can include, be for, be performed for, apply, correspond to, be diagnostic of (e.g., for diagnosing, etc.), be therapeutic of (e.g., therapeutic composition including epitopes therapeutic of, etc.), and/or otherwise be associated with one or more conditions, including any one or more of: diseases, symptoms, causes (e.g., triggers, etc.), disorders, associated risk (e.g., propensity scores, etc.), associated severity, behaviors (e.g., caffeine consumption, habits, diets, etc.), and/or any other suitable aspects associated with conditions. Conditions can include one or more disease-related conditions, which can include any one or more of: gastrointestinal-related conditions (e.g., irritable bowel syndrome, inflammatory bowel disease, ulcerative colitis, celiac disease, Crohn's disease, bloating, hemorrhoidal disease, constipation, reflux, bloody stool, diarrhea, etc.); allergy-related conditions (e.g., allergies and/or intolerance associated with wheat, gluten, dairy, soy, peanut, shellfish, tree nut, egg, etc.); skin-related conditions (e.g., acne, dermatomyositis, eczema, rosacea, dry skin, psoriasis, dandruff, photosensitivity, etc.); locomotor-related conditions (e.g., gout, rheumatoid arthritis, osteoarthritis, reactive arthritis, multiple sclerosis, Parkinson's disease, etc.); cancer-related conditions (e.g., lymphoma; leukemia; blastoma; germ cell tumor; carcinoma; sarcoma; breast cancer; prostate cancer; basal cell cancer; skin cancer; colon cancer; lung cancer; cancer conditions associated with any suitable physiological region; etc.), cardiovascular-related conditions (e.g., coronary heart disease, inflammatory heart disease, valvular heart disease, obesity, stroke, etc.), anemia conditions (e.g., thalassemia; sickle cell; pernicious; fanconi; haemolyitic; aplastic; iron deficiency; etc.), neurological-related conditions (e.g., ADHD, ADD, anxiety, Asperger's syndrome, autism, chronic fatigue syndrome, depression, etc.), autoimmune-related conditions (e.g., Sprue, AIDS, Sjogren's, Lupus, etc.), endocrine-related conditions (e.g., obesity, Graves' disease, Hashimoto's thyroiditis, metabolic disease, Type I diabetes, Type II diabetes, etc.), Lyme disease conditions, communication-related conditions, sleep-related conditions, metabolic-related conditions, weight-related conditions, pain-related conditions, genetic-related conditions, chronic disease, and/or any other suitable type of disease-related conditions. Additionally or alternatively, microorganism-related conditions can include one or more human behavior conditions which can include any one or more of: caffeine consumption, alcohol consumption, other food item consumption, dietary supplement consumption, probiotic-related behaviors (e.g., consumption, avoidance, etc.), other dietary behaviors, habitué behaviors (e.g., smoking; exercise conditions such as low, moderate, and/or extreme exercise conditions; etc.), menopause, other biological processes, social behavior, other behaviors, and/or any other suitable human behavior conditions. Conditions can be associated with any suitable phenotypes (e.g., phenotypes measurable for a human, animal, plant, fungi body, etc.).

In some embodiments, the present disclosures provides a method (e.g., a workflow) to identify “de novo” epitopes and/or reengineered ones from proteins belonging to one or more pathogens (e.g., described herein; other suitable microorganisms), and/or searching them in inversely-associated bacteria, such as to propose them as new epitope-based vaccines and/or suitable therapeutic compositions, intended to trigger an immune response, and/or for diagnostics, therapeutics, and/or prevention of any one or more conditions described herein, such as based on approaches described herein.

In some embodiments, the present disclosures provides a method for identifying epitopes from non-pathogenic microorganisms (e.g., any suitable type of microorganisms, virus, bacteria, archaea, etc.), such as based off of epitopes derived from pathogenic microorganisms or agents that produce an immunological response; such as finding non-pathogenic microorganism epitopes that are similar, reengineered, and/or analogous to pathogenic microorganism epitopes; such as for facilitating improved safety in relation to epitope usage in diagnostics and/or therapeutics; such as based on using approaches described herein.

### P89-PRV1-6

In some embodiments, the disclosure provides a method to predict de novo epitopes against HPV, based on Lactobacillus species present in a healthy vaginal microbiome. Such epitope-based approach provides a method for HPV treatment and detection. In some embodiments, the disclosure provides a system, and therapeutic compositions using the epitope-based approach for HPV treatment and detection.

A widely used strategy to identify T cell epitopes includes at least first predicting HLA binding peptides by in-silico methods. As an example, we first consider the HPV proteomes of strains 6, 11 (involved in genital warts), and 16, 18 (involved in cervical cancer), specifically proteins E1, E2, E4, E5, E6, E7, L1 and L2, and additionally E8 protein for type 11, 16 and 18 to obtain a list of predicted 8-9 amino acids peptides. Those servers have in common that they are able to predict peptides which bind human leukocyte antigen (HLA) class I allele B, that correspond to the human version of the major histocompatibility complex (MHC). HLA complex can present those peptide antigens as epitopes.

In specific examples, once the list of de novo epitopes has been obtained and repetitive sequences removed, 148 Lactobacillus reference proteomes of different species and one Fusobacterium nucleatum reference proteome were downloaded from protein sequences databases, such as Uniprot; each protein sequence and each epitope were aligned by using local pairwise alignment tools, in order to find “de novo” predicted epitopes in those proteomes.

Thus, in specific examples, sequences found in Lactobacillus and Fusobacterium can be considered as “common epitopes” compared to the predicted HPV epitopes, according the following criteria:

1. Sequences having more than 70% identity and 100% matches*, and
2. Sequences having 100% identity and more or equal than 7 matches*,
where “match” is the local similarity of an amino acid position in a pairwise alignment.

However, any suitable criteria (e.g., any suitable percent identity, percent matches, number of matches, any suitable percent similarity such as 60% similarity, etc.) can be used.

Common epitopes sequences that are part of associated bacteria described in literature, (e.g., that are directly involved in the triggering of diseases, such as Gardnerella vaginalis, Chlamydia trachomatis and Sneathia, etc.) were discarded; but any suitable filtering criteria can additionally or alternatively be used.

Finally, in specific examples, to classify “common epitopes” according their affinity to the receptor, we tested our database of 155 filtered de novo epitopes from HPV 6, 11, 16 and 18 using molecular docking simulations.

Accordingly, in a specific example, a general view of the workflow employed to identify “de novo” epitopes from pathogen proteins associated to a particular condition, and then search them in proteomes from inversely-associated organisms, can be summarized in FIG. 7 (e.g., including variations of an embodiment).

As a specific example, the workflow depicted in FIG. 7 can be applied to for the search of “de novo” epitopes in HPV proteomes and then, in Lactobacillus proteomes, as described above.

The use of predicted epitopes from any strains of HPV or reengineered ones, and/or other suitable microorganisms, in different products for the diagnostics, treatment and/or prevention of HPV and/or suitable conditions described herein, such as based on approaches described herein.

In some embodiments, the disclosure provides therapeutic compositions including one or more epitopes described herein, such as epitopes derived from microorganisms described herein and/or other suitable microorganisms, such as for use in diagnostics, therapeutics, and/or other suitable applications, such as in relation to one or more conditions (e.g., cancer, warts, etc.) described herein, such as based on approaches described herein.

In some embodiments, the disclosure provides a method for diagnostics and/or therapeutic compositions using “de novo” predicted epitopes and/or reengineered ones, such as derived from microorganisms described herein and/or other suitable microorganism for the treatment and/or prevention of HPV and/or suitable conditions described herein, such as based on approaches described herein.

In some embodiments, the disclosure provides a method (e.g., a workflow) to identify “de novo” epitopes and/or reengineered ones from proteins belonging to one or more pathogens (e.g., described herein; other suitable microorganisms), and/or searching them in inversely-associated bacteria, such as to propose them as new epitope-based vaccines and/or suitable therapeutic compositions, intended to trigger an immune response, and/or for diagnostics, therapeutics, and/or prevention of any one or more conditions described herein, such as based on approaches described herein.

In some embodiments, the disclosure provides a method for identifying epitopes from non-pathogenic microorganisms (e.g., any suitable type of microorganisms, virus, bacteria, archaea, etc.), such as based off of epitopes derived from pathogenic microorganisms, such as finding non-pathogenic microorganism epitopes that are similar, reengineered, and/or analogous to pathogenic microorganism epitopes, such as for facilitating improved safety in relation to epitope usage in diagnostics and/or therapeutics, such as based on using approaches described herein.

### HPV Type 6 (HPV6) Infection

In some embodiments, the disclosure provides therapeutic compositions including one or more epitopes described herein, such as epitopes derived from microorganisms described herein and/or other suitable microorganisms, for diagnosing, treating, or preventing HPV6 infection.

In some embodiments, the disclosure provides a method for diagnostics and/or therapeutic compositions using “de novo” predicted epitopes and/or reengineered ones, such as derived from microorganisms described herein and/or other suitable microorganism for the treatment and/or prevention of HPV6.

In some embodiments, the disclosure provides a method (e.g., a workflow) to identify “de novo” epitopes and/or reengineered ones from proteins belonging to one or more pathogens (e.g., described herein; other suitable microorganisms), and/or searching them in inversely-associated bacteria, such as to propose them as new epitope-based vaccines and/or suitable therapeutic compositions, intended to trigger an immune response, for the treatment and/or prevention of HPV6.

In some embodiments, the disclosure provides a method for identifying epitopes from non-pathogenic microorganisms (e.g., any suitable type of microorganisms, virus, bacteria, archaea, etc.), such as based off of epitopes derived from pathogenic microorganisms, such as finding non-pathogenic microorganism epitopes that are similar, reengineered, and/or analogous to pathogenic microorganism epitopes, such as for facilitating improved safety in relation to epitope usage in the treatment and/or prevention of HPV6.

In some embodiments, the disclosure provides a method for treating or preventing HPV6 infection comprising administering to a patient in need thereof a polypeptide comprising: (a) an epitope comprising the sequence (and/or suitable portion of the sequence, such as at least 7, optionally at least 8 contiguous amino acids): IGAAIGYFY (from Lactobacillus oryzae and L. malefermentans) and/or GTAGILELL (from Lactobacillus sp-wkB8); (b) an epitope comprising at least 8 contiguous amino acids (and/or suitable portion of the sequence) of at least one of LVLTLLLYL and/or SVLVLTLLL; (c) an epitope comprising at least 7, optionally at least 8, contiguous amino acids of at least one of DPYKNLSFW, CAFIVGVLG, RTGISNAST, DSNVRLVVQ, VVLPDPNKF, ISFLGGTVI, VQIAAGTTS, HCYEQLVDS, KHAIVTVTY, KAKQMGLSH, KNALTTAEI, MEAIAKRLD, NTMDYVVWT, TSSETTTPA, VARTLATLL, PNNGKYVMA, NVVKIPPTI, APTITSHPI, PAVSKASAA, NNGKYVMAA, YPDYLQMAA, PVQIAAGTT, KQDILDVLI, TVETTTSSL, PVFITGSGF, HPYFSIKRA, TVQDLKRKY, MESANASTS, PTQHPVTNI, NSHLATPCV, TVARTLATL, IPPTIRHKL, LLLTTPLQF, VLGLLLMHY, QIAAGTTST, RKHKALTLI, VVCFVSIIL, TVVPKVSGY, NGKYVMAAQ, and/or SRARRRKRA; and/or (e) an epitope of the sequence IGARIHYFY comprising one or more substitutions (e.g., Position 1 substituted with A, E, H, or V; Position 2 substituted with Y; Position 3 substituted with S; Position 4 substituted with Y, A, L, F, H, or P; Position 5 substituted with N, D, A, T, or Y; Position 6 substituted with Q; and/or Position 8 substituted with P). Additionally or alternatively, the (one or more) polypeptides can comprise any epitope or combination of epitopes from (a), (b) and/or (c), whereby any epitope with an amino acid substitution in any position of the epitope can be made, such as while maintaining properties and/or improving affinity with the HLA receptor.

In an embodiment, the polypeptide comprises a Lactobacillus and/or Fusobacterium polypeptide, and/or fragment thereof, such as comprising the one or more epitopes. In an embodiment, the polypeptide comprises a 10-amino acid or larger fragment of a Lactobacillus and/or Fusobacterium polypeptide, and optionally, wherein the fragment is smaller or equal to 100 amino acids (however, the fragment and/or polypeptide can be of any suitable size, such as including any suitable number of amino acids).

In an embodiment, the bacterium comprises Lactobacillus sp., such as preferably L. acetotolerans, L. agilis, L. alimentarius, L. backii, L. bifermentans, L. brevis, L. cacaonum, L. coleohominis, L. coleohominis-DSM, L. collinoides, L. concavus, L. coryniformis, L. diolivorans, L. equigenerosi, L. fabifermentans, L. farraginis, L. fermentum, L. florum, L. frumenti, L. fuchuensis, L. ingluviei, L. johnsonii, L. kimchicus, L. koreensis, L. lindneri, L. mali, L. manihotivorans, L. mellis, L. nodensis, L. odoratitofui, L. oeni, L. oligofermentans, L. olsenella-uli, L. oris-PB013-T2-3, L. oryzae, L. parabrevis, L. parafarraginis, L. plantarum, L. rennini, L. rossiae, L. ruminis, L. sanfranciscensis, L. saniviri, L. satchensis, L. secaliphilus, L. senioris, L. sharpeae, L. suebicus, L. tucceti, L. vaccinostercus, L. vini, L. viridescens, L. wasatchensis, and/or L. zymae.

In an embodiment, the polypeptide is administered to the patient by administering a cell (one or more cells) expressing the polypeptide(s), wherein the cell(s) can be genetically modified to overexpress the polypeptide(s). The cell(s) can be (e.g., associated with) a bacterium, such as preferably from the genera Lactobacillus and/or Fusobacterium.

In an embodiment, the polypeptide(s) is administered to the patient by transforming a cell(s) of the patient with a nucleic acid(s) encoding the polypeptide(s), wherein the nucleic acid is optionally in a vector(s). In variations, the cell(s) can be transformed ex vivo and the transformed cell(s) re-introduced into the patient. In variations, the cell(s) can be transformed in vivo by administering the nucleic acid(s), optionally in a vector(s), to the patient.

In an embodiment, one or more isolated polypeptides, the polypeptide comprising: (a) an epitope comprising the sequence: IGAAIGYFY (from Lactobacillus oryzae and L. malefermentans and/or GTAGILELL (from Lactobacillus sp-wkB8); (b) an epitope comprising at least 8 contiguous amino acids of at least one of LVLTLLLYL and/or SVLVLTLLL; (c) an epitope comprising at least 7, optionally at least 8, contiguous amino acids of at least one of DPYKNLSFW, CAFIVGVLG, RTGISNAST, DSNVRLVVQ, VVLPDPNKF, ISFLGGTVI, VQIAAGTTS, HCYEQLVDS, KHAIVTVTY, KAKQMGLSH, KNALTTAEI, MEAIAKRLD, NTMDYVVWT, TSSETTTPA, VARTLATLL, PNNGKYVMA, NVVKIPPTI, APTITSHPI, PAVSKASAA, NNGKYVMAA, YPDYLQMAA, PVQIAAGTT, KQDILDVLI, TVETTTSSL, PVFITGSGF, HPYFSIKRA, TVQDLKRKY, MESANASTS, PTQHPVTNI, NSHLATPCV, TVARTLATL, IPPTIRHKL, LLLTTPLQF, VLGLLLMHY, QIAAGTTST, RKHKALTLI, VVCFVSIIL, TVVPKVSGY, NGKYVMAAQ, and/or SRARRRKRA; and/or (d) an epitope of the sequence IGARIHYFY comprising one or more substitutions (e.g., Position 1 substituted with A, E, H, or V; Position 2 substituted with Y; Position 3 substituted with S; Position 4 substituted with Y, A, L, F, H, or P; Position 5 substituted with N, D, A, T, or Y; Position 6 substituted with Q; and/or Position 8 substituted with P), and/or PVFITGSDF comprising one or more substitutions (e.g., Position 1 substituted with W or F; Position 2 substituted with F, D, or W; Position 5 substituted with P; Position 6 substituted with P, A, or C; Position 7 substituted with W, Q, F, or P; Position 8 substituted with W; and/or Position 9 substituted with W).

In some embodiments, the disclosure provides a pharmaceutical composition(s) comprising a pharmaceutically acceptable carrier(s) and one or more polypeptide described herein for treating or preventing HPV6 infection.

In some embodiments, the disclosure provides a polypeptide comprising a sequence, or a sequence having at least 80% identity, e.g., at least 85%, 90%, 95%, 99% identity, to a sequence selected from the group consisting of IGAAIGYFY, GTAGILELL, LVLTLLLYL, SVLVLTLLL, DPYKNLSFW, CAFIVGVLG, RTGISNAST, DSNVRLVVQ, VVLPDPNKF, ISFLGGTVI, VQIAAGTTS, HCYEQLVDS, KHAIVTVTY, KAKQMGLSH, KNALTTAEI, MEAIAKRLD, NTMDYVVWT, TSSETTTPA, VARTLATLL, PNNGKYVMA, NVVKIPPTI, APTITSHPI, PAVSKASAA, NNGKYVMAA, YPDYLQMAA, PVQIAAGTT, KQDILDVLI, TVETTTSSL, PVFITGSGF, HPYFSIKRA, TVQDLKRKY, MESANASTS, PTQHPVTNI, NSHLATPCV, TVARTLATL, IPPTIRHKL, LLLTTPLQF, VLGLLLMHY, QIAAGTTST, RKHKALTLI, VVCFVSIIL, TVVPKVSGY, NGKYVMAAQ, and SRARRRKRA.

In some embodiments, the disclosure provides a polypeptide comprising a sequence, or a sequence having at least 80% identity, e.g., at least 85%, 90%, 95%, 99% identity, to the sequence of X₁X₂X₃X₄X₅X₆X₇X₈X₉, wherein:

X₁ is I, A, E, H, or V;

X₂ is G or Y;

X₃ is A or S;

X₄ is R, Y, A, L, F, H, or P;

X₅ is I, N, D, A, T, or Y;

X₆ is H or O;

X₇ is Y;

X₈ is F or P;

X₉ is Y.

In some embodiments, the disclosure provides a polypeptide comprising a sequence, or a sequence having at least 80% identity, e.g., at least 85%, 90%, 95%, 99% identity, to the sequence of X₁X₂X₃X₄X₅X₆X₇X₈X₉, wherein:

X₁ is P, W or F;

X₂ is V, F, D, or W;

X₃ is F;

X₄ is I;

X₅ is T or P;

X₆ is G, P, A, or C;

X₇ is S, W, Q, F, or P;

X₈ is D or W;

X₉ is F or W.

In some embodiments, the disclosure provides a method for treating or preventing HPV6 infection comprising administering to a patient in need thereof a pharmaceutical composition comprising the polypeptide having a sequence, or a sequence having at least 80% identity, e.g., at least 85%, 90%, 95%, 99% identity, to a sequence selected from the group consisting of IGAAIGYFY, GTAGILELL, LVLTLLLYL, SVLVLTLLL, DPYKNLSFW, CAFIVGVLG, RTGISNAST, DSNVRLVVQ, VVLPDPNKF, ISFLGGTVI, VQIAAGTTS, HCYEQLVDS, KHAIVTVTY, KAKQMGLSH, KNALTTAEI, MEAIAKRLD, NTMDYVVWT, TSSETTTPA, VARTLATLL, PNNGKYVMA, NVVKIPPTI, APTITSHPI, PAVSKASAA, NNGKYVMAA, YPDYLQMAA, PVQIAAGTT, KQDILDVLI, TVETTTSSL, PVFITGSGF, HPYFSIKRA, TVQDLKRKY, MESANASTS, PTQHPVTNI, NSHLATPCV, TVARTLATL, IPPTIRHKL, LLLTTPLQF, VLGLLLMHY, QIAAGTTST, RKHKALTLI, VVCFVSIIL, TVVPKVSGY, NGKYVMAAQ, and SRARRRKRA.

In some embodiments, the disclosure provides a method for treating or preventing HPV6 infection comprising administering to a patient in need thereof a pharmaceutical composition comprising the polypeptide having a sequence, or a sequence having at least 80% identity, e.g., at least 85%, 90%, 95%, 99% identity, to the sequence of X₁X₂X₃X₄X₅X₆X₇X₈X₉, wherein:

X₁ is I, A, E, H, or V;

X₂ is G or Y;

X₃ is A or S;

X₄ is R, Y, A, L, F, H, or P;

X₅ is I, N, D, A, T, or Y;

X₆ is H or O;

X₇ is Y;

X₈ is F or P;

X₉ is Y.

In some embodiments, the disclosure provides a method for treating or preventing HPV6 infection comprising administering to a patient in need thereof a pharmaceutical composition comprising the polypeptide having a sequence, or a sequence having at least 80% identity, e.g., at least 85%, 90%, 95%, 99% identity, to the sequence of X₁X₂X₃X₄X₅X₆X₇X₈X₉, wherein:

X₁ is P, W or F;

X₂ is V, F, D, or W;

X₃ is F;

X₄ is I;

X₅ is T or P;

X₆ is G, P, A, or C;

X₇ is S, W, Q, F, or P;

X₈ is D or W;

X₉ is F or W.

### HPV Type 11 (HPV11) Infection

In some embodiments, the disclosure provides therapeutic compositions including one or more epitopes described herein, such as epitopes derived from microorganisms described herein and/or other suitable microorganisms, for diagnosing, treating, or preventing HPV11 related diseases.

In some embodiments, the disclosure provides a method for diagnostics and/or therapeutic compositions using “de novo” predicted epitopes and/or reengineered ones, such as derived from microorganisms described herein and/or other suitable microorganism for the treatment and/or prevention of HPV11.

In some embodiments, the disclosure provides a method (e.g., a workflow) to identify “de novo” epitopes and/or reengineered ones from proteins belonging to one or more pathogens (e.g., described herein; other suitable microorganisms), and/or searching them in inversely-associated bacteria, such as to propose them as new epitope-based vaccines and/or suitable therapeutic compositions, intended to trigger an immune response, for the treatment and/or prevention of HPV11.

In some embodiments, the disclosure provides a method for identifying epitopes from non-pathogenic microorganisms (e.g., any suitable type of microorganisms, virus, bacteria, archaea, etc.), such as based off of epitopes derived from pathogenic microorganisms, such as finding non-pathogenic microorganism epitopes that are similar, reengineered, and/or analogous to pathogenic microorganism epitopes, such as for facilitating improved safety in relation to epitope usage in the treatment and/or prevention of HPV11.

Embodiments (e.g., of one or more methods, of one or more systems, such as compositions, etc.) can additionally or alternatively include:

In some embodiments, the disclosure provides a method for treating or preventing HPV 11 infection comprising administering to a patient in need thereof a polypeptide comprising: (a) an epitope comprising the sequence (and/or suitable portion of the sequence, such as at least 7, optionally at least 8 contiguous amino acids) of at least one of PVFITGSDF, LSTPQRLVT, FVVAVLGLL, TPFSPVTPA, LPVVIAFAV, LVAAENDTF, PSDSTVYVP, GAPEVVPPT, SDSTVYVPP, QGNTVEVKF, LVLTLLLYL, RVGLYSRAL, LILPVVIAF, TSSESTTPA, DSNVRLVVE, VLIILISDF, KPRARRRKR, VQIAAATTT, RRLFETREL, LTDAKVALL, MADDSALYE, EVVPVQIAA, NAVYELSDA, SSESTTPAI, QIAAATTTT, KIQSGVRAL, TVARTLGTL, STSATSIDQ, TSSLTITTS, ETNEDILKV, TVQSTTSSL, RALQQVQVT, LKDIVLDLQ, PVQIAAATT, YSIKKVNKT, PRARRRKRA, ASTSATSID, RKHRALTLI, ADDSALYEK, KCKDIRSTL, STTSSLTIT, RKTACRRRL, VVIAFAVCI, AIAKRLDAC, and/or MEVVPVQIA, and/or (b) an epitope of the sequence PVFITGSDF comprising one or more substitutions (e.g., Position 1 substituted with W or F; Position 2 substituted with F, D, or W; Position 5 substituted with P; Position 6 substituted with P, A, or C; Position 7 substituted with W, Q, F, or P; Position 8 substituted with W; Position 9 substituted with W). Additionally or alternatively, the (one or more) polypeptides can comprise any epitope or combination of epitopes from (a), (b) and/or (c), whereby any epitope with an amino acid substitution in any position of the epitope can be made, such as while maintaining properties and/or improving affinity with the HLA receptor.

In some embodiments, the polypeptide comprises a Lactobacillus and/or Fusobacterium polypeptide, and/or fragment thereof, such as comprising the one or more epitopes.

In some embodiments, the polypeptide comprises a 10-amino acid or larger fragment of a Lactobacillus and/or Fusobacterium polypeptide, and optionally, wherein the fragment is smaller or equal to 100 amino acids (however, the fragment and/or polypeptide can be of any suitable size, such as including any suitable number of amino acids).

In some embodiments, the bacterium comprises Lactobacillus sp., such as preferably L. acetotolerans, L. acidophilus, L. algidus, L. alimentarius, L. amylolyticus, L. amylophilus, L. apodemi, L. aviarius, L. backii, L. brantae, L. cacaonum, L. capillatus, L. coleohominis, L. concavus, L. coryniformis, L. crispatus, L. delbrueckii, L. dextrinicus, L. equi, L. fabifermentans, L. floricola, L. florum, L. frumenti, L. ghanensis, L. hamsteri, L. harbinensis, L. helveticus, L. hokkaidonensis, L. hominis, L. iners, L. jensenii, L. johnsonii, L. kalixensis, L. kefiranofaciens, L. kimchicus, L. kisonensis, L. kunkeei, L. lindneri, L. malefermentans, L. mellifer, L. nasuensis, L. nodensis, L. odoratitofui, L. olsenella-uli, L. parafarraginis, L. pasteurii, L. perolens, L. phage-phiAQ113, L. plantarum, L. rhamnosus, L. rossiae, L. ruminis, L. sakei, L. saniviri, L. selangorensis, L. senmaizukei, L. sharpeae, L. similis, L. sp-ASF360, L. sp-wkB8, L. tucceti, L. vini, L. viridescens, L. xiangfangensis, and/or L. zymae.

In some embodiments, the polypeptide is administered to the patient by administering a cell (one or more cells) expressing the polypeptide(s), wherein the cell(s) can be genetically modified to overexpress the polypeptide(s). The cell(s) can be (e.g., associated with) a bacterium, such as preferably from the genera Lactobacillus and/or Fusobacterium.

In some embodiments, the polypeptide(s) is administered to the patient by transforming a cell(s) of the patient with a nucleic acid(s) encoding the polypeptide(s), wherein the nucleic acid is optionally in a vector(s). In variations, the cell(s) can be transformed ex vivo and the transformed cell(s) re-introduced into the patient. In variations, the cell(s) can be transformed in vivo by administering the nucleic acid(s), optionally in a vector(s), to the patient.

In some embodiments, the polypeptide comprises: (a) an epitope comprising at least 7, optionally at least 8, contiguous amino acids of at least one of PVFITGSDF, LSTPQRLVT, FVVAVLGLL, TPFSPVTPA, LPVVIAFAV, LVAAENDTF, PSDSTVYVP, GAPEVVPPT, SDSTVYVPP, QGNTVEVKF, LVLTLLLYL, RVGLYSRAL, LILPVVIAF, TSSESTTPA, DSNVRLVVE, VLIILISDF, KPRARRRKR, VQIAAATTT, RRLFETREL, LTDAKVALL, MADDSALYE, EVVPVQIAA, NAVYELSDA, SSESTTPAI, QIAAATTTT, KIQSGVRAL, TVARTLGTL, STSATSIDQ, TSSLTITTS, ETNEDILKV, TVQSTTSSL, RALQQVQVT, LKDIVLDLQ, PVQIAAATT, YSIKKVNKT, PRARRRKRA, ASTSATSID, RKHRALTLI, ADDSALYEK, KCKDIRSTL, STTSSLTIT, RKTACRRRL, VVIAFAVCI, AIAKRLDAC, and/or MEVVPVQIA; and/or (b) an epitope of the sequence PVFITGSDF comprising one or more substitutions (e.g., Position 1 substituted with W or F; Position 2 substituted with F, D, or W; Position 5 substituted with P; Position 6 substituted with P, A, or C; Position 7 substituted with W, Q, F, or P; Position 8 substituted with W; Position 9 substituted with W).

In some embodiments, the disclosure provides a pharmaceutical composition for treating or preventing HPV11 infection comprising a pharmaceutically acceptable carrier(s) and one or more polypeptide as described herein.

In some embodiments, the disclosure provides a polypeptide comprising the sequence having at least 80% identity to a sequence selected from the group consisting of PVFITGSDF, LSTPQRLVT, FVVAVLGLL, TPFSPVTPA, LPVVIAFAV, LVAAENDTF, PSDSTVYVP, GAPEVVPPT, SDSTVYVPP, QGNTVEVKF, LVLTLLLYL, RVGLYSRAL, LILPVVIAF, TSSESTTPA, DSNVRLVVE, VLIILISDF, KPRARRRKR, VQIAAATTT, RRLFETREL, LTDAKVALL, MADDSALYE, EVVPVQIAA, NAVYELSDA, SSESTTPAI, QIAAATTTT, KIQSGVRAL, TVARTLGTL, STSATSIDQ, TSSLTITTS, ETNEDILKV, TVQSTTSSL, RALQQVQVT, LKDIVLDLQ, PVQIAAATT, YSIKKVNKT, PRARRRKRA, ASTSATSID, RKHRALTLI, ADDSALYEK, KCKDIRSTL, STTSSLTIT, RKTACRRRL, VVIAFAVCI, AIAKRLDAC, and MEVVPVQIA.

In some embodiments, the disclosure provides a polypeptide comprising a sequence, or a sequence having at least 80% identity, e.g., at least 85%, 90%, 95%, 99% identity, to the sequence of X₁₀X₁₁X₁₂X₁₃X₁₄X₁₅X₁₆X₁₇X₁₈, wherein:

X₁₀ is P, W, or F;

X₁₁ is V, F, D, or W;

X₁₂ is F;

X₁₃ is I;

X₁₄ is T or P;

X₁₅ is G, P, A, or C;

X₁₆ is S, W, Q, F, or P;

X₁₇ is D or W;

X₁₈ is F or W.

In some embodiments, the disclosure provides a method for treating or preventing HPV11 infection comprising administering to a patient in need thereof a pharmaceutical composition comprising the polypeptide comprising a sequence, or a sequence having at least 80% identity, e.g., at least 85%, 90%, 95%, 99% identity, to a sequence selected from the group consisting of PVFITGSDF, LSTPQRLVT, FVVAVLGLL, TPFSPVTPA, LPVVIAFAV, LVAAENDTF, PSDSTVYVP, GAPEVVPPT, SDSTVYVPP, QGNTVEVKF, LVLTLLLYL, RVGLYSRAL, LILPVVIAF, TSSESTTPA, DSNVRLVVE, VLIILISDF, KPRARRRKR, VQIAAATTT, RRLFETREL, LTDAKVALL, MADDSALYE, EVVPVQIAA, NAVYELSDA, SSESTTPAI, QIAAATTTT, KIQSGVRAL, TVARTLGTL, STSATSIDQ, TSSLTITTS, ETNEDILKV, TVQSTTSSL, RALQQVQVT, LKDIVLDLQ, PVQIAAATT, YSIKKVNKT, PRARRRKRA, ASTSATSID, RKHRALTLI, ADDSALYEK, KCKDIRSTL, STTSSLTIT, RKTACRRRL, VVIAFAVCI, AIAKRLDAC, and MEVVPVQIA.

In some embodiments, the disclosure provides a method for treating or preventing HPV11 infection comprising administering to a patient in need thereof a pharmaceutical composition comprising the polypeptide comprising a sequence, or a sequence having at least 80% identity, e.g., at least 85%, 90%, 95%, 99% identity, to the sequence of X₁₀X₁₁X₁₂X₁₃X₁₄X₁₅X₁₆X₁₇X₁₈, wherein:

X₁₀ is P, W, or F;

X₁₁ is V, F, D, or W;

X₁₂ is F;

X₁₃ is I;

X₁₄ is T or P;

X₁₅ is G, P, A, or C;

X₁₆ is S, W, Q, F, or P;

X₁₇ is D or W;

X₁₈ is F or W.

### HPV Type 16 (HPV16) Infection

In some embodiments, the disclosure provides therapeutic compositions including one or more epitopes described herein, such as epitopes derived from microorganisms described herein and/or other suitable microorganisms, for diagnosing, treating, or preventing HPV16 related diseases.

In some embodiments, the disclosure provides a method for diagnostics and/or therapeutic compositions using “de novo” predicted epitopes and/or reengineered ones, such as derived from microorganisms described herein and/or other suitable microorganism for the treatment and/or prevention of HPV16.

In some embodiments, the disclosure provides a method (e.g., a workflow) to identify “de novo” epitopes and/or reengineered ones from proteins belonging to one or more pathogens (e.g., described herein; other suitable microorganisms), and/or searching them in inversely-associated bacteria, such as to propose them as new epitope-based vaccines and/or suitable therapeutic compositions, intended to trigger an immune response, for the treatment and/or prevention of HPV16.

In some embodiments, the disclosure provides a method for identifying epitopes from non-pathogenic microorganisms (e.g., any suitable type of microorganisms, virus, bacteria, archaea, etc.), such as based off of epitopes derived from pathogenic microorganisms, such as finding non-pathogenic microorganism epitopes that are similar, reengineered, and/or analogous to pathogenic microorganism epitopes, such as for facilitating improved safety in relation to epitope usage in the treatment and/or prevention of HPV16.

In some embodiments, the disclosure provides a method for treating or preventing HPV type 16 infection comprising administering to a patient in need thereof a polypeptide comprising: (a) an epitope comprising the sequence (and/or suitable portion of the sequence, such as at least 7, optionally at least 8 contiguous amino acids): LLKLVGSTS (from Lactobacillus Acidophilus), LLTLLGSPW (from Lactobacillus floricola), LLMLLGLTW (from Lactobacillus plantarum), LGKWLGSTW (from Lactobacillus nasuensis), LAKLLGSGW (from Lactobacillus sakei); (b) an epitope comprising at least 8 contiguous amino acids (and/or suitable portion of the sequence) of at least one of SAFLKSNSQ, PPTPAETGG, and/or VEKKTGDAI; (c) an epitope comprising at least 7, optionally at least 8, contiguous amino acids of at least one of FWLQPLADA, QPPTPAETG, SGKSIGAKV, PYLHNRLVV, AHTKDGLTV, VAVNPGDCP, SHAASPTSI, TPAILDINN, AEEIELQTI, NALDGNLVS, FELSQMVQW, IAEQILQYG, GGLGIGTGS, TAHALFTAQ, VPTLAVSKN, ADPAAATKY, YDLSTIDPA, AGTSRLLAV, NASAFLKSN, TLCQRLNVC, HAASPTSIN, YSLYGTTLE, LGKRKATPT, CEEASVTVV, LWLPSEATV, IPIVPGSPQ, MADPAAATK, KPYWLQRAQ, DPAGTNGEE, LAKFKELYG, IGNKQTLRT, GNQLFVTVV, DAGDFYLHP, YGNTEVETQ, TPPRPIPKP, AAMLAKFKE, PFDENGNPV, LGIGTGSGT, ATKYPLLKL, GEDLVDFIV, INHQVVPTL, TPSIADSIK, ICEEASVTV, LYLHIQSLA, LADTNSNAS, DYLTQAETE, SLIPIVPGS, and/or RAAKRRLFE; and/or (d) an epitope of the sequence FWLQPLADA comprising one or more substitutions (e.g., Position 1 substituted with W, Y, A, or I; Position 2 substituted with Y, F, G, I, L, A, C, S, R, T, Q, or V; Position 3 substituted with N, D, W, F, P, Q, V, I, E, R, A, C, T, Y, H, or S; Position 4 substituted with D, P, N, S, A, E, L, F, C, W, H, I, T, G, or K; Position 5 substituted with D or H; Position 6 substituted with E, N, F, Y, G, P, T, D, A, Q, I, V, or H; Position 7 substituted with W, F, Y, E, Q, V, G, L, P, or M; Position 8 substituted with Y, T, A, W, P, F, S, Q, E, K, R, H, V, or C; Position 9 substituted with F, L, W, P, H, V, S, N, C, E, or M). Additionally or alternatively, the (one or more) polypeptides can comprise any epitope or combination of epitopes from (a), (b), (c) and/or (d), whereby any epitope with an amino acid substitution in any position of the epitope can be made, such as while maintaining properties and/or improving affinity with the HLA receptor.

In some embodiments, the polypeptide comprises a Lactobacillus and/or Fusobacterium polypeptide, and/or fragment thereof, such as comprising the one or more epitopes.

In some embodiments, the polypeptide comprises a 10-amino acid or larger fragment of a Lactobacillus and/or Fusobacterium polypeptide, and optionally, wherein the fragment is smaller or equal to 100 amino acids (however, the fragment and/or polypeptide can be of any suitable size, such as including any suitable number of amino acids).

In some embodiments, the bacterium comprises Lactobacillus sp., such as preferably L. acetotolerans, L. agilis, L. algidus, L. amylophilus, L. bifermentans, L. brevis, L. brevis-subsp-gravesensis, L. buchneri, L. capillatus, L. collinoides, L. composti, L. crispatus, L. curieae, L. delbrueckii, L. diolivorans, L. fabifermentans, L. farraginis, L. gastricus, L. ginsenosidimutans, L. hamsteri, L. harbinensis, L. hominis, L. jensenii, L. kisonensis, L. koreensis, L. kunkeei, L. malefermentans, L. mucosae-LM1, L. odoratitofui, L. olsenella-uli, L. parabrevis, L. parafarraginis, L. rossiae, L. ruminis, L. saniviri, L. senmaizukei, L. casei, L. sharpeae, L. shenzhenensis, L. siliginis, L. similis, L. sp-wkB8, L. spicheri, L. suebicus, L. tucceti, L. wasatchensis, L. xiangfangensis, and/or L. zymae.

In some embodiments, the polypeptide is administered to the patient by administering a cell (one or more cells) expressing the polypeptide(s), wherein the cell(s) can be genetically modified to overexpress the polypeptide(s). The cell(s) can be (e.g., associated with) a bacterium, such as preferably from the genera Lactobacillus and/or Fusobacterium.

In some embodiments, the polypeptide(s) is administered to the patient by transforming a cell(s) of the patient with a nucleic acid(s) encoding the polypeptide(s), wherein the nucleic acid is optionally in a vector(s). In variations, the cell(s) can be transformed ex vivo and the transformed cell(s) re-introduced into the patient. In variations, the cell(s) can be transformed in vivo by administering the nucleic acid(s), optionally in a vector(s), to the patient.

In some embodiments, the disclosure provides one or more isolated polypeptides, the polypeptide comprising: (a) an epitope comprising the sequence: LLKLVGSTS (from Lactobacillus Acidophilus), LLTLLGSPW (from Lactobacillus floricola), LLMLLGLTW (from Lactobacillus plantarum), LGKWLGSTW (from Lactobacillus nasuensis), LAKLLGSGW (from Lactobacillus sakei); (b) an epitope comprising at least 8 contiguous amino acids of at least one of SAFLKSNSQ, PPTPAETGG, and/or VEKKTGDAI; (c) an epitope comprising at least 7, optionally at least 8, contiguous amino acids of at least one of FWLQPLADA, QPPTPAETG, SGKSIGAKV, PYLHNRLVV, AHTKDGLTV, VAVNPGDCP, SHAASPTSI, TPAILDINN, AEEIELQTI, NALDGNLVS, FELSQMVQW, IAEQILQYG, GGLGIGTGS, TAHALFTAQ, VPTLAVSKN, ADPAAATKY, YDLSTIDPA, AGTSRLLAV, NASAFLKSN, TLCQRLNVC, HAASPTSIN, YSLYGTTLE, LGKRKATPT, CEEASVTVV, LWLPSEATV, IPIVPGSPQ, MADPAAATK, KPYWLQRAQ, DPAGTNGEE, LAKFKELYG, IGNKQTLRT, GNQLFVTVV, DAGDFYLHP, YGNTEVETQ, TPPRPIPKP, AAMLAKFKE, PFDENGNPV, LGIGTGSGT, ATKYPLLKL, GEDLVDFIV, INHQVVPTL, TPSIADSIK, ICEEASVTV, LYLHIQSLA, LADTNSNAS, DYLTQAETE, SLIPIVPGS, and/or RAAKRRLFE; and/or (d) an epitope of the sequence FWLQPLADA comprising one or more substitutions (e.g., Position 1 substituted with W, Y, A, or I; Position 2 substituted with Y, F, G, I, L, A, C, S, R, T, Q, or V; Position 3 substituted with N, D, W, F, P, Q, V, I, E, R, A, C, T, Y, H, or S; Position 4 substituted with D, P, N, S, A, E, L, F, C, W, H, I, T, G, or K; Position 5 substituted with D or H; Position 6 substituted with E, N, F, Y, G, P, T, D, A, Q, I, V, or H; Position 7 substituted with W, F, Y, E, Q, V, G, L, P, or M; Position 8 substituted with Y, T, A, W, P, F, S, Q, E, K, R, H, V, or C; Position 9 substituted with F, L, W, P, H, V, S, N, C, E, or M).

In some embodiments, the disclosure provides a pharmaceutical composition(s) comprising a pharmaceutically acceptable carrier(s) and one or more polypeptide as described herein.

In some embodiments, the disclosure provides a polypeptide comprising the sequence having at least 80% identity to a sequence selected from the group consisting of LLKLVGSTS, LLTLLGSPW, LLMLLGLTW, LGKWLGSTW, LAKLLGSGW, SAFLKSNSQ, PPTPAETGG, VEKKTGDAI, FWLQPLADA, QPPTPAETG, SGKSIGAKV, PYLHNRLVV, AHTKDGLTV, VAVNPGDCP, SHAASPTSI, TPAILDINN, AEEIELQTI, NALDGNLVS, FELSQMVQW, IAEQILQYG, GGLGIGTGS, TAHALFTAQ, VPTLAVSKN, ADPAAATKY, YDLSTIDPA, AGTSRLLAV, NASAFLKSN, TLCQRLNVC, HAASPTSIN, YSLYGTTLE, LGKRKATPT, CEEASVTVV, LWLPSEATV, IPIVPGSPQ, MADPAAATK, KPYWLQRAQ, DPAGTNGEE, LAKFKELYG, IGNKQTLRT, GNQLFVTVV, DAGDFYLHP, YGNTEVETQ, TPPRPIPKP, AAMLAKFKE, PFDENGNPV, LGIGTGSGT, ATKYPLLKL, GEDLVDFIV, INHQVVPTL, TPSIADSIK, ICEEASVTV, LYLHIQSLA, LADTNSNAS, DYLTQAETE, SLIPIVPGS, and RAAKRRLFE.

In some embodiments, the disclosure provides a polypeptide comprising a sequence, or a sequence having at least 80% identity, e.g., at least 85%, 90%, 95%, 99% identity, to the sequence of X₁₉X₂₀X₂₁X₂₂X₂₃X₂₄X₂₅X₂₆X₂₇, wherein:

X₁₉ is F, W, Y, A, or I;

X₂₀ is W, Y, F, G, I, L, A, C, S, R, T, Q, or V;

X₂₁ is L, N, D, W, F, P, Q, V, I, E, R, A, C, T, Y, H, or S;

X₂₂ is Q, D, P, N, S, A, E, L, F, C, W, H, I, T, G, or K;

X₂₃ is P, D, or H;

X₂₄ is L, E, N, F, Y, G, P, T, D, A, Q, I, V, or H;

X₂₅ is A, W, F, Y, E, Q, V, G, L, P, or M;

X₂₆ is D, Y, T, A, W, P, F, S, Q, E, K, R, H, V, or C;

X₂₇ is A, F, L, W, P, H, V, S, N, C, E, or M.

In some embodiments, the disclosure provides a method for treating or preventing HPV16 infection comprising administering to a patient in need thereof a pharmaceutical composition comprising the polypeptide comprising a sequence selected from the group consisting of LLKLVGSTS, LLTLLGSPW, LLMLLGLTW, LGKWLGSTW, LAKLLGSGW, SAFLKSNSQ, PPTPAETGG, VEKKTGDAI, FWLQPLADA, QPPTPAETG, SGKSIGAKV, PYLHNRLVV, AHTKDGLTV, VAVNPGDCP, SHAASPTSI, TPAILDINN, AEEIELQTI, NALDGNLVS, FELSQMVQW, IAEQILQYG, GGLGIGTGS, TAHALFTAQ, VPTLAVSKN, ADPAAATKY, YDLSTIDPA, AGTSRLLAV, NASAFLKSN, TLCQRLNVC, HAASPTSIN, YSLYGTTLE, LGKRKATPT, CEEASVTVV, LWLPSEATV, IPIVPGSPQ, MADPAAATK, KPYWLQRAQ, DPAGTNGEE, LAKFKELYG, IGNKQTLRT, GNQLFVTVV, DAGDFYLHP, YGNTEVETQ, TPPRPIPKP, AAMLAKFKE, PFDENGNPV, LGIGTGSGT, ATKYPLLKL, GEDLVDFIV, INHQVVPTL, TPSIADSIK, ICEEASVTV, LYLHIQSLA, LADTNSNAS, DYLTQAETE, SLIPIVPGS, and RAAKRRLFE.

In some embodiments, the disclosure provides a method for treating or preventing HPV16 infection comprising administering to a patient in need thereof a pharmaceutical composition comprising the polypeptide or a polypeptide having at least 80% identity, e.g., at least 85%, 90%, 95%, 99% identity, to the sequence of X₁₉X₂₀X₂₁X₂₂X₂₃X₂₄X₂₅X₂₆X₂₇, wherein:

X₁₉ is F, W, Y, A, or I;

X₂₀ is W, Y, F, G, I, L, A, C, S, R, T, Q, or V;

X₂₁ is L, N, D, W, F, P, Q, V, I, E, R, A, C, T, Y, H, or S;

X₂₂ is Q, D, P, N, S, A, E, L, F, C, W, H, I, T, G, or K;

X₂₃ is P, D, or H;

X₂₄ is L, E, N, F, Y, G, P, T, D, A, Q, I, V, or H;

X₂₅ is A, W, F, Y, E, Q, V, G, L, P, or M;

X₂₆ is D, Y, T, A, W, P, F, S, Q, E, K, R, H, V, or C;

X₂₇ is A, F, L, W, P, H, V, S, N, C, E, or M.

### HPV Type 18 (HPV18) Infection

In some embodiments, the disclosure provides therapeutic compositions including one or more epitopes described herein, such as epitopes derived from microorganisms described herein and/or other suitable microorganisms, for diagnosing, treating, or preventing HPV18 related diseases.

In some embodiments, the disclosure provides a method for diagnostics and/or therapeutic compositions using “de novo” predicted epitopes and/or reengineered ones, such as derived from microorganisms described herein and/or other suitable microorganism for the treatment and/or prevention of HPV18.

In some embodiments, the disclosure provides a method (e.g., a workflow) to identify “de novo” epitopes and/or reengineered ones from proteins belonging to one or more pathogens (e.g., described herein; other suitable microorganisms), and/or searching them in inversely-associated bacteria, such as to propose them as new epitope-based vaccines and/or suitable therapeutic compositions, intended to trigger an immune response, for the treatment and/or prevention of HPV18.

In some embodiments, the disclosure provides a method for identifying epitopes from non-pathogenic microorganisms (e.g., any suitable type of microorganisms, virus, bacteria, archaea, etc.), such as based off of epitopes derived from pathogenic microorganisms, such as finding non-pathogenic microorganism epitopes that are similar, reengineered, and/or analogous to pathogenic microorganism epitopes, such as for facilitating improved safety in relation to epitope usage in the treatment and/or prevention of HPV18.

In some embodiments, the disclosure provides a method for treating or preventing HPV18 infection comprising administering to a patient in need thereof a polypeptide comprising: (a) an epitope comprising the sequence (and/or suitable portion of the sequence, such as at least 7, optionally at least 8 contiguous amino acids): QKQLEILGC (from Lactobacillus floricola); (b) an epitope comprising at least 8 contiguous amino acids (and/or suitable portion of the sequence) of at least one of GGQTVQVYF and/or QATTKDGNS; (c) an epitope comprising at least 7, optionally at least 8, contiguous amino acids of at least one of KNGNPVYEI, HRFSTSDDT, KGGQTVQVY, KSRLTVAKG, ICGHYIILF, QHRFSTSDD, KQGAMLAVF, KAHKAIELQ, SIVDLSTHF, and/or ETLSERLSC; and/or (d) an epitope of the sequence GGQTVQVYF comprising one or more substitutions (e.g., Position 1 substituted with W, F, Y, P, R, C, I or L; Position 2 substituted with P, D, F, A, Q, Y or S; Position 3 substituted with W, Y, H, R, V, F, L, P, A, D, G or S; Position 4 substituted with W, A, F, P, G, H, R, Y, D, N, Q or S; Position 5 substituted with W, G or T; Position 6 substituted with N, E, G, P or W; Position 7 substituted with D, T or A; Position 8 substituted with D, W or F; Position 9 substituted with W). Additionally or alternatively, the (one or more) polypeptides can comprise any epitope or combination of epitopes from (a), (b), (c), and/or (d), whereby any epitope with an amino acid substitution in any position of the epitope can be made, such as while maintaining properties and/or improving affinity with the HLA receptor.

In some embodiments, the polypeptide comprises a Lactobacillus and/or Fusobacterium polypeptide, and/or fragment thereof, such as comprising the one or more epitopes.

In some embodiments, the polypeptide comprises a 10-amino acid or larger fragment of a Lactobacillus and/or Fusobacterium polypeptide, and optionally, wherein the fragment is smaller or equal to 100 amino acids (however, the fragment and/or polypeptide can be of any suitable size, such as including any suitable number of amino acids).

In some embodiments, the bacterium comprises Lactobacillus sp., such as preferably L. floricola, L. crispatus, L. similis, L. suebicus, L. oris-PB013-T2-3, L. johnsonii, L. perolens, L. bifermentans, L. collinoides, L. plantarum, and/or L. brevis subsp-gravesensis.

In some embodiments, the polypeptide is administered to the patient by administering a cell (one or more cells) expressing the polypeptide(s), wherein the cell(s) can be genetically modified to overexpress the polypeptide(s). The cell(s) can be (e.g., associated with) a bacterium, such as preferably from the genera Lactobacillus and/or Fusobacterium.

In some embodiments, the polypeptide(s) is administered to the patient by transforming a cell(s) of the patient with a nucleic acid(s) encoding the polypeptide(s), wherein the nucleic acid is optionally in a vector(s). In variations, the cell(s) can be transformed ex vivo and the transformed cell(s) re-introduced into the patient. In variations, the cell(s) can be transformed in vivo by administering the nucleic acid(s), optionally in a vector(s), to the patient.

In some embodiments, the disclosure provides one or more isolated polypeptides, the polypeptide comprising: (a) an epitope comprising the sequence: QKQLEILGC (from Lactobacillus floricola); (b) an epitope comprising at least 8 contiguous amino acids of at least one of GGQTVQVYF and/or QATTKDGNS; (c) an epitope comprising at least 7, optionally at least 8, contiguous amino acids of at least one of KNGNPVYEI, HRFSTSDDT, KGGQTVQVY, KSRLTVAKG, ICGHYIILF, QHRFSTSDD, KQGAMLAVF, KAHKAIELQ, SIVDLSTHF, and/or ETLSERLSC; and/or (d) an epitope of the sequence GGQTVQVYF comprising one or more substitutions (e.g., Position 1 substituted with W, F, Y, P, R, C, I or L; Position 2 substituted with P, D, F, A, Q, Y or S; Position 3 substituted with W, Y, H, R, V, F, L, P, A, D, G or S; Position 4 substituted with W, A, F, P, G, H, R, Y, D, N, Q or S; Position 5 substituted with W, G or T; Position 6 substituted with N, E, G, P or W; Position 7 substituted with D, T or A; Position 8 substituted with D, W or F; Position 9 substituted with W).

In some embodiments, the disclosure provides a pharmaceutical composition(s) comprising a pharmaceutically acceptable carrier(s) and one or more polypeptide described herein.

In some embodiments, the disclosure provides a polypeptide comprising a sequence, or a sequence having at least 80% identity, e.g., at least 85%, 90%, 95%, 99% identity, to a sequence selected from the group consisting of QKQLEILGC, GGQTVQVYF, QATTKDGNS, KNGNPVYEI, HRFSTSDDT, KGGQTVQVY, KSRLTVAKG, ICGHYIILF, QHRFSTSDD, KQGAMLAVF, KAHKAIELQ, SIVDLSTHF, and ETLSERLSC.

In some embodiments, the disclosure provides a polypeptide comprising a sequence, or a sequence having at least 80% identity, e.g., at least 85%, 90%, 95%, 99% identity, to the sequence of X₂₈X₂₉X₃₀X₃₁X₃₂X₃₃X₃₄X₃₅X₃₆, wherein:

X₂₈ is G, W, F, Y, P, R, C, I or L;

X₂₉ is G, P, D, F, A, Q, Y or S;

X₃₀ is Q, W, Y, H, R, V, F, L, P, A, D, G or S;

X₃₁ is T, W, A, F, P, G, H, R, Y, D, N, Q or S;

X₃₂ is V, W, G or T;

X₃₃ is Q, N, E, G, P or W;

X₃₄ is V, D, T or A;

X₃₅ is Y, D, W or F;

X₃₆ is F or W.

In some embodiments, the disclosure provides a method for treating or preventing HPV18 infection comprising administering to a patient in need thereof a pharmaceutical composition comprising the polypeptide comprising a sequence, or a sequence having at least 80% identity, e.g., at least 85%, 90%, 95%, 99% identity, to a sequence selected from the group consisting of QKQLEILGC, GGQTVQVYF, QATTKDGNS, KNGNPVYEI, HRFSTSDDT, KGGQTVQVY, KSRLTVAKG, ICGHYIILF, QHRFSTSDD, KQGAMLAVF, KAHKAIELQ, SIVDLSTHF, and ETLSERLSC.

In some embodiments, the disclosure provides a method for treating or preventing HPV18 infection comprising administering to a patient in need thereof a pharmaceutical composition comprising the polypeptide comprising a sequence, or a sequence having at least 80% identity, e.g., at least 85%, 90%, 95%, 99% identity, to the sequence of X₂₈X₂₉X₃₀X₃₁X₃₂X₃₃X₃₄X₃₅X₃₆, wherein:

X₂₈ is G, W, F, Y, P, R, C, I or L;

X₂₉ is G, P, D, F, A, Q, Y or S;

X₃₀ is Q, W, Y, H, R, V, F, L, P, A, D, G or S;

X₃₁ is T, W, A, F, P, G, H, R, Y, D, N, Q or S;

X₃₂ is V, W, G or T;

X₃₃ is Q, N, E, G, P or W;

X₃₄ is V, D, T or A;

X₃₅ is Y, D, W or F;

X₃₆ is F or W.

Embodiments of the method can, however, include any other suitable blocks or steps configured to facilitate reception of biological samples from subjects, processing of biological samples from subjects, analyzing data derived from biological samples, and generating models that can be used to provide customized diagnostics and/or probiotic-based therapeutics according to specific microbiome compositions and/or functional features of subjects.

Embodiments of the method and/or system can include every combination and permutation of the various system components and the various method processes, including any variants (e.g., embodiments, variations, examples, specific examples, figures, etc.), where portions of embodiments of the method and/or processes described herein can be performed asynchronously (e.g., sequentially), concurrently (e.g., in parallel), or in any other suitable order by and/or using one or more instances, elements, components of, and/or other aspects of the system and/or other entities described herein.

Any of the variants described herein (e.g., embodiments, variations, examples, specific examples, figures, etc.) and/or any portion of the variants described herein can be additionally or alternatively combined, aggregated, excluded, used, performed serially, performed in parallel, and/or otherwise applied.

Portions of embodiments of the method and/or system can be embodied and/or implemented at least in part as a machine configured to receive a computer-readable medium storing computer-readable instructions. The instructions can be executed by computer-executable components that can be integrated with the system. The computer-readable medium can be stored on any suitable computer-readable media such as RAMs, ROMs, flash memory, EEPROMs, optical devices (CD or DVD), hard drives, floppy drives, or any suitable device. The computer-executable component can be a general or application specific processor, but any suitable dedicated hardware or hardware/firmware combination device can alternatively or additionally execute the instructions.

As a person skilled in the art will recognize from the previous detailed description and from the figures and claims, modifications and changes can be made to embodiments of the method, system, and/or variants without departing from the scope defined in the claims.

### P123-PRV

Trimethylamine (TMA), produced by the gut microbiota from dietary quaternary amines (mainly choline and L-carnitine). TMA is later converted within the body to trimethylamine N-oxide (TMAO). TMAO has been associated with reduced cardiovascular wellness, and is associated with atherosclerosis and severe cardiovascular disease. People with higher levels of TMAO in their blood tend to be at risk for arterial plaque buildup. Currently, little information on the composition of TMA producers in the gut is available due to their low abundance and the requirement of specific functional-based detection methods as many taxa show disparate abilities to produce that compound.

Choline and L-carnitine are found in many foods, especially beef, pork, and lamb. L-carnitine also is found in eggs.

In some embodiments, after the intake of red meat and eggs, some of the gut microbes convert compounds found in these foods into a chemical called trimethylamine (TMA). Your body then converts TMA to trimethylamine N-oxide (TMAO).

The disclosure provides a method of preventing or treating cardiovascular disease. Such method comprises changing your intake of choline or L-carnitine via changing diet. In some embodiments, the disclosure provides a method of preventing or treating cardiovascular disease by changing to Mediterranean diet, choline-controlled diet, vegan or vegetarian diet, or Resveratrol Supplements.

### Mediterranean Diet

This type of diet consists primarily of fresh fruits and vegetables, plant-derived oils (such as olive oil), seeds, nuts, fish, and beans. It is low in saturated fats (such as butter), dairy, and red meat.

Goal: Limit the consumption of choline and L-carnitine, compounds found especially in red meat that can be converted to TMA.

### Choline-Controlled Diet

Try a diet that avoids excess choline, which is most highly concentrated in beef and eggs. This diet includes soy protein beverages, five daily servings of vegetables and fruits, bread, grains and cereals, and low amounts of fats and oils.

### Vegan or Vegetarian Diet

Adopt one of these diets. Vegans and vegetarians have been shown to have less of the compound L-carnitine and fewer of the microbes that produce TMA.

### Resveratrol Supplements

Try taking resveratrol supplements. Resveratrol is a natural polyphenol plant compound that limits the production of TMA by microbes in your gut.

### EXAMPLES

### Example 1: Identification of Epitopes from the Proteomes of HPV

As an example of the method described herein, epitopes from the proteomes of HPV type 6 (777 sequences), 11 (719 sequences), 16 (1278 sequences) and 18 (740 sequences) were identified.

From those sequences, 3514 epitopes from T cell prediction servers were predicted. Once repetitive sequences were discarded, only 823 “de novo” epitopes resulted, which were aligned against proteins belonging to 148 species of Lactobacillus and Fusobacterium nucleatum. Consequently, 155 “common epitopes” between HPV and Lactobacillus proteins and 1 epitope in common between HPV and Fusobacterium nucleatum proteins were obtained. Those epitopes that did not fit with the criteria described herein were discarded.

Regarding the 156 “de novo” epitopes, 5 of them also belong to bacteria known to be associated with HPV infection. They are: RAAKRRLFE epitope from HPV 16 and L. crispatus, GGQTVQVYF and KGGQTVQVY epitopes from HPV 18 and L. crispatus, QIAAATTTT epitope from HPV 11 and L. iners and L. crispatus, and KNALTTAEI from HPV 6 and Fusobacterium nucleatum. The latter was also found in three species of Lactobacillus: equigenerosi, fuchuensis and odoratitofui.

As an example, those 156 peptide sequences as epitopes against HPV infection to prevent cervical cancer and genital warts were tested. The list of epitopes is shown in the next tables (Table 1 and Table 2). Additionally, the energy of binding from docking simulations, expressed in kcal/mol, is also listed. The energy of binding can be understood as a ranking to determine epitopes with a higher affinity (lower energy of binding) for the HLA class I receptor.

TABLE 1
List of “de novo” predicted epitopes suggested for protection against HPV infection and
prevention of cervical cancer (HPV type 16 and 18).
A) Filter 1: Predicted HPV epitopes found in bacteria with more than 70% of sequence identity
and 100% matches.
PREVENTION OF CERVICAL CANCER

| HPV16 | | | HPV18 | | |
|---|---|---|---|---|---|
| | Energy of | | | Energy of | |
| | binding | | | binding | |
| Epitope | (kcal/mol) | Lactobacillus sp. | Epitope | (kcal/mol) | Lactobacillus sp. |
| LLKLLGST | −8.5 | Acidophilus (LLKLVGSTS), | QTQLCILGC | −7.5 | Floricola (QKQLEILGC) |
| W | | floricola (LLTLLGSPW), | | | |
| | | nasuensis (LGKWLGSTW), | | | |
| | | plantarum (LLMLLGLTW), | | | |
| | | sakei (LAKLLGSGW) | | | |

B) Filter 2: Predicted HPV epitopes found in bacteria with 100% of sequence identity and 8
matches.
PREVENTION OF CERVICAL CANCER

| HPV16 | | | HPV18 | | |
|---|---|---|---|---|---|
| | Energy of | | | Energy of | |
| | binding | | | binding | |
| Epitope | (kcal/mol) | Lactobacillus sp. | Epitope | (kcal/mol) | Lactobacillus sp. |
| SAFLKSNSQ | −8.1 | tucceti | GGQTVQVYF | −9.2 | crispatus |
| PPTPAETGG | −8 | parafarraginis | QATTKDGNS | −7.8 | similis |
| VEKKTGDAI | −7.4 | casei | | | |

C) Filter 3: Predicted HPV epitopes found in bacteria with 100% of sequence identity and 7
matches.
HPV STRAINS ASSOCIATED TO PREVENTION OF CERVICAL CANCER

| HPV16 | | | HPV18 | | |
|---|---|---|---|---|---|
| | Energy of | | | Energy of | |
| | binding | | | binding | |
| Epitope | (kcal/mol) | Lactobacillus sp. | Epitope | (kcal/mol) | Lactobacillus sp. |
| FWLQPLADA | −9.7 | mucosae-LM1, harbinensis, | KNGNPVYEI | −9.1 | suebicus |
| | | kisonensis, | | | |
| | | shenzhenensis, suebicus, | | | |
| | | malefermentans | | | |
| VAVNPGDCP | -9.4 | farraginis, spicheri, brevis, | KGGQTVQVY | −8.7 | crispatus |
| | | senmaizukeicasei | | | |
| FELSQMVQW | −9.2 | buchneri | ICGHYIILF | −8.7 | oris-PB013-T2-3 |
| ADPAAATKY | −9.2 | koreensis | KQGAMLAVF | −8.6 | johnsonii |
| HAASPTSIN | −9.2 | hominis | SIVDLSTHF | −8.4 | perolens |
| LWLPSEATV | −9 | xiangfangensis | HRFSTSDDT | −8.2 | bifermentans |
| DPAGTNGEE | −9 | rossiae | KSRLTVAKG | −8.1 | collinoides |
| DAGDFYLHP | −9 | agilis | QHRFSTSDD | −8.1 | bifermentans |
| PFDENGNPV | −8.9 | tucceti, mucosae-LM1, | KAHKAIELQ | −7.6 | plantarum |
| | | hamsteri, jensenii, | | | |
| | | odoratitofui, sp-wkB8 | | | |
| INHQVVPTL | −8.8 | harbinensis | ETLSERLSC | -5.9 | brevis-subsp-gravesensis |
| LADTNSNAS | −8.8 | mucosae-LM1 | | | |
| QPPTPAETG | −8.7 | parafarraginis | | | |
| SHAASPTSI | −8.7 | hominis | | | |
| IAEQILQYG | −8.7 | algidus | | | |
| YDLSTIDPA | −8.6 | gastricus | | | |
| YSLYGTTLE | −8.5 | siliginis | | | |
| IPIVPGSPQ | −8.5 | wasatchensis | | | |
| LAKFKELYG | −8.5 | amylophilus | | | |
| YGNTEVETQ | −8.5 | bifermentans | | | |
| LGIGTGSGT | −8.4 | collinoides, spicheri, | | | |
| | | diolivorans, koreensis, | | | |
| | | zymae, brevis, similis, | | | |
| | | parabrevis | | | |
| TPSIADSIK | −8.3 | spicheri, koreensis | | | |
| DYLTQAETE | −8.1 | ruminis | | | |
| SGKSIGAKV | −8.1 | acetotolerans | | | |
| TPAILDINN | −8.1 | suebicus | | | |
| GGLGIGTGS | −8.1 | spicheri, wasatchensis, | | | |
| | | koreensis, zymae, brevis, | | | |
| | | parabrevis | | | |
| AGTSRLLAV | −8 | fabifermentans | | | |
| LGKRKATPT | −8 | malefermentans | | | |
| MADPAAATK | −8 | koreensis | | | |
| IGNKQTLRT | −8 | malefermentans | | | |
| TPPRPIPKP | −7.9 | olsenella-uli | | | |
| ATKYPLLKL | −7.9 | curieae | | | |
| ICEEASVTV | −7.9 | jensenii | | | |
| SLIPIVPGS | −7.9 | wasatchensis | | | |
| PYLHNRLVV | −7.9 | buchneri | | | |
| AEEIELQTI | −7.8 | ginsenosidimutans | | | |
| TAHALFTAQ | −7.8 | zymae, capillatus | | | |
| NASAFLKSN | −7.8 | tucceti | | | |
| CEEASVTVV | −7.7 | jensenii | | | |
| KPYWLQRAQ | −7.6 | brevis | | | |
| GNQLFVTVV | −7.6 | saniviri | | | |
| AAMLAKFKE | −7.6 | sharpeae | | | |
| GEDLVDFIV | −7.6 | parafarraginis, farraginis, | | | |
| | | brevis-subsp-gravesensis | | | |
| LYLHIQSLA | −7.5 | rossiae | | | |
| RAAKRRLFE | −7.5 | crispatus | | | |
| AHTKDGLTV | −7.4 | kunkeei, xiangfangensis, | | | |
| | | composti | | | |
| NALDGNLVS | −7.4 | curieae, delbrueckii | | | |
| VPTLAVSKN | −7.2 | sharpeae | | | |
| TLCQRLNVC | −7.2 | koreensis | | | |

The epitopes are classified according docking energy of binding expressed in kcal/mol. Additionally, the column of Lactobacillus sp contains the name of the species where the epitope was found.

TABLE 2
List of de novo epitopes suggested for prevention of genital warts (HPV type 6 and 11).
A) Filter 1: Predicted HPV epitopes found in bacteria with more than 70% of sequence identity
and 100% matches.
PREVENTION OF GENITAL WARTS

| HPV6 | | | HPV11 | | |
|---|---|---|---|---|---|
| | Energy of | | | Energy of | |
| | binding | | | binding | |
| Epitope | (kcal/mol) | Lactobacillus sp. | Epitope | (kcal/mol) | Lactobacillus sp. |
| IGARIHYFY | −9.8 | oryzae (IGAAIGYFY), | — | — | — |
| | | malefermentans | | | |
| | | (IGAAIGYFY) | | | |
| GTAGILELL | −8.1 | sp-wkB8 (GTAG1LELL) | — | — | — |

B) Filter 2: Predicted HPV epitopes found in bacteria with 100% of sequence identity and 8
matches.
PREVENTION OF GENITAL WARTS

| HPV6 | | | HPV11 | | |
|---|---|---|---|---|---|
| | Energy of | | | Energy of | |
| | binding | | | binding | |
| Epitope | (kcal/mol) | Lactobacillus sp. | Epitope | (kcal/mol) | Lactobacillus sp. |
| LVLTLLLYL | −8.5 | capillatus, apodemi, | — | — | — |
| | | dextrinicus | | | |
| SVLVLTLLL | −7.8 | coryniformis | — | — | — |
| | | fuchuensis, apodemi, | | | |
| | | camelliae, composti, | | | |
| | | thailandensis, vini | | | |

C) Filter 3: Predicted HPV epitopes found in bacteria with 100% of sequence identity and 7
matches.
PREVENTION OF GENITAL WARTS

| HPV6 | | | HPV11 | | |
|---|---|---|---|---|---|
| | Energy of | | | Energy of | |
| | binding | | | binding | |
| Epitope | (kcal/mol) | Lactobacillus sp. | Epitope | (kcal/mol) | Lactobacillus sp. |
| DPYKNLSFW | −9.6 | wasatchensis | PVFITGSDF | −9.5 | hokkaidonensis, |
| | | | | | plantarum, tucceti |
| VVLPDPNKF | −9.2 | johnsonii | LSTPQRLVT | −9.4 | perolens |
| KHAIVTVTY | −9.1 | fabifermentans | FVVAVLGLL | −9.4 | malefermentans, |
| | | | | | senmaizukei |
| NTMDYVVW | −9.1 | brevis | TPFSPVTPA | −9.2 | similis |
| T | | | | | |
| NVVKIPPTI | −9 | plantarum | LPVVIAFAV | −9.1 | backii |
| YPDYLQMAA | −9 | agilis | LVAAENDTF | −9.1 | parafarraginis |
| PVFITGSGF | −9 | ruminis | PSDSTVYVP | −8.9 | floricola |
| PTQHPVTNI | −8.8 | sanfranciscensis, backii, | GAPEVVPPT | −8.9 | harbinensis, viridescens |
| | | florum, lindneri, oeni | | | |
| LLLTTPLQF | −8.7 | rennini | SDSTVYVPP | −8.8 | floricola |
| VVCFVSIIL | −8.7 | agilis, ruminis | QGNTVEVKF | −8.8 | vini |
| CAFIVGVLG | −8.6 | collinoides | LVLTLLLYL | −8.7 | capillatus, apodemi, |
| | | | | | dexfrinicus |
| ISFLGGTVI | −8.5 | vini | RVGLYSRAL | −8.6 | equi |
| KAKQMGLSH | −8.4 | diolivorans, parafarraginis | LILPVVIAF | −8.5 | lindneri |
| TSSETTTPA | −8.4 | backii | TSSESTTPA | −8.4 | algidus, apodemi |
| APTITSHPI | −8.4 | koreensis | DSNVRLVVE | −8.4 | lindneri |
| PVQIAAGTT | −8.3 | ingluviei | VLIILISDF | −8.4 | amylophilus, |
| | | | | | hokkaidonensis, kimchicus, |
| | | | | | odoratitofui, rhamnosus |
| HPYFSIKRA | −8.3 | florum | KPRARRRKR | −8.3 | olsenella-uli |
| NSHLATPCV | −8.2 | kimchicus | VQIAAATTT | −8.3 | ghanensis, nodensis |
| VLGLLLMHY | −8.2 | diolivorans, florum, rennini, | RRLFETREL | −8.3 | selangorensis |
| | | rossiae | | | |
| TVVPKVSGY | −8.1 | nodensis | LTDAKVALL | −8.3 | fabifermentans, similis |
| RTGISNAST | −8.1 | sharpeae | MADDSALYE | −8.2 | zymae |
| VQIAAGTTS | −8 | ingluviei | EVVPVQIAA | −8.2 | plantarum, ruminis |
| KNALTTAEI | −8 | equigenerosi, fuchuensis, | NAVYELSDA | −8.2 | coryniformis |
| | | odoratitofui | | | |
| VARTLATLL | −8 | plantarum, senioris | SSESTTPAI | −8.2 | mellifer |
| PAVSKASAA | −7.9 | bifermentans, coryniformis, | QIAAATTTT | −8.1 | acetotolerans, acidophilus, |
| | | fermentum, rennini, saniviri, | | | amylolyticus, brantae, |
| | | tucceti | | | coleohominis, concavus, |
| | | | | | crispatus, delbrueckii, |
| | | | | | dextrinicus, hamsteri, |
| | | | | | helveticus, hominis, iners, |
| | | | | | jensenii, johnsonii, |
| | | | | | kalixensis, kefiranofaciens, |
| | | | | | kunkeei, mellifer, |
| | | | | | nasuensis, nodensis, |
| | | | | | pasteurii, sakei, saniviri, |
| | | | | | selangorensis, sp-ASF360, |
| | | | | | sp-wkB8, xiangfangensis |
| KQDILDVLI | −7.9 | diolivorans | KIQSGVRAL | −8.1 | kisonensis |
| TVQDLKRKY | −7.9 | plantarum | TVARTLGTL | −8.1 | acidophilus, crispatus |
| TVARTLATL | −7.9 | plantarum | STSATSIDQ | −8 | fabifermentans |
| QIAAGTTST | −7.8 | parabrevis | TSSLTITTS | −8 | florum |
| NGKYVMAA | −7.8 | acetotolerans | ETNEDILKV | −8 | nodensis, tucceti |
| Q | | | | | |
| DSNVRLVVQ | −7.8 | lindneri | TVQSTTSSL | −7.9 | odoratitofui |
| HCYEQLVDS | −7.8 | fermentum | RALQQVQVT | −7.9 | fabifermentans |
| MEAIAKRLD | −7.7 | alimentarius, coleohominis, | LKDIVLDLQ | −7.8 | senmaizukei |
| | | coleohominis-DSH, | | | |
| | | concavus, coryniformis, | | | |
| | | diolivorans, farraginis, | | | |
| | | fermentum, frumenti, | | | |
| | | manihotivorans, | | | |
| | | oligofermentans, oris- | | | |
| | | PB013-T2-3, oryzae, rennini, | | | |
| | | secaliphilus, suebicus, | | | |
| | | vaccinostercus, viridescens, | | | |
| | | wasatchensis, zymae | | | |
| PNNGKYVMA | −7.7 | acetotolerans | PVQIAAATT | −7.8 | nodensis |
| NNGKYVMA | −7.7 | acetotolerans | YSIKKVNKT | −7.8 | apodemi |
| A | | | | | |
| TVETTTSSL | −7.6 | vaccinostercus | PRARRRKRA | −7.7 | olsenella-uli |
| MESANASTS | −7.5 | oryzae | ASTSATSID | −7.6 | fabifermentans |
| IPPTIRHKL | −7.5 | mellis | RKHRALTLI | −7.5 | sharpeae |
| RKHKALTLI | −7.2 | cacaonum, frumenti, mali | ADDSALYEK | −7.3 | zymae |
| SRARRRKRA | −6.7 | olsenella-uli | KCKDIRSTL | −7.3 | jensenii |
| | | | STTSSLTIT | −7.2 | cacaonum |
| | | | RKTACRRRL | −7.1 | aviarius |
| | | | VVIAFAVCI | −7 | backii, phage-phiAQ113 |
| | | | AIAKRLDAC | −7 | alimentarius, helveticus, |
| | | | | | rossiae |
| | | | MEVVPVQIA | −7 | plantarum, ruminis |
| | | | TAPTVSACT | −6.8 | frumenti |

Epitopes are classified according to docking energy of binding expressed in kcal/mol. Additionally, the column of Lactobacillus sp contains the species where the epitope was found.

In a next stage, in a variation, to improve the affinity of epitopes for the receptor, it is also possible to subject the best epitopes to a reengineering, which means that every amino acid can be mutated in-silico, one at the time, by the other 19 amino acids. Those new epitopes obtained by reengineering can be tested by docking and/or other suitable techniques, and then classified according their energy of binding to the receptor. In this way, it is possible to obtain new epitopes with a better affinity to the receptor.

Thus, as another example, a preferred set of epitopes (e.g., with best docking energy score), obtained from, but not limited to each different HPV type (6, 11, 16 and 18) were selected for reengineering. In addition, as shown in Tables 3 to 6, the first row represents the amino acid composition of each one of those wild-type (WT) epitopes. Next rows are representing the 20 amino acids to which each position was mutated, and the respective docking energy values, being represented only those having equal or higher affinity (e.g lower energy) than obtained for each WT epitope (showed in the table header). Below Tables 3-6, mutations improving the affinity of each epitope are disclosed.

TABLE 3
Reengineering of the best epitope from HPV-6.
IGARIHYFY peptide (WT energy: −9.8 kcal/mol)

| | ILE1 | GLY2 | ALA3 | ARG4 | ILE5 | HIS6 | TYR7 | PHE8 | TYR9 |
|---|---|---|---|---|---|---|---|---|---|
| ALA | −10 | | | −10 | −9.8 | | | | |
| CYS | | | | | | | | | |
| ASP | | | | | −10 | | | | |
| GLU | −9.9 | | | | | | | | |
| PHE | | | | −9.8 | | | | | |
| GLY | | | | | | | | | |
| HIS | −9.8 | | | −9.8 | | | | | |
| ILE | | | | | | | | | |
| LYS | | | | | | | | | |
| LEU | | | | −10 | | | | | |
| MET | | | | | | | | | |
| ASN | | | | | −10.2 | | | | |
| PRO | | | | −9.8 | | | | −9.8 | |
| GLN | | | | | | −9.9 | | | |
| ARG | | | | | | | | | |
| SER | | | −9.9 | | | | | | |
| THR | | | | | −9.8 | | | | |
| VAL | −9.8 | | | | | | | | |
| TRP | | | | | | | | | |
| TYR | | −10.3 | | −10.2 | −9.8 | | | | |

IGARIHYFY and its mutations at the corresponding position:
1: A, E, H, or V
2: Y
3: S
4: Y, A, L, F, H, or P
5: N, D, A, T, or Y
6: Q
8: P

TABLE 4
Reengineering of the best epitope from HPV-11.
PVFITGSDF peptide (WT energy: −9.5 kcal/mol)

| | PRO1 | VAL2 | PHE3 | ILE4 | THR5 | GLY6 | SER7 | ASP8 | PHE9 |
|---|---|---|---|---|---|---|---|---|---|
| ALA | | | | | | −9.6 | | | |
| CYS | | | | | | −9.5 | | | |
| ASP | | −9.5 | | | | | | | |
| GLU | | | | | | | | | |
| PHE | −9.6 | −9.6 | | | | | −9.6 | | |
| GLY | | | | | | | | | |
| HIS | | | | | | | | | |
| ILE | | | | | | | | | |
| LYS | | | | | | | | | |
| LEU | | | | | | | | | |
| MET | | | | | | | | | |
| ASN | | | | | | | | | |
| PRO | | | | | −10.0 | −9.8 | −9.5 | | |
| GLN | | | | | | | −9.7 | | |
| ARG | | | | | | | | | |
| SER | | | | | | | | | |
| THR | | | | | | | | | |
| VAL | | | | | | | | | |
| TRP | −9.9 | −9.5 | | | | | −9.9 | −9.7 | −9.9 |
| TYR | | | | | | | | | |

PVFITGSDF and its mutations at the corresponding position:
1: W, or F
2: F, D, or W
5: P
6: P, A, or C
7: W, Q, F, or P
8: W
9: W

TABLE 5
Reengineering of the best epitope from HPV-16.
FWLQPLADA peptide (WT energy: −9.7 kcal/mol)

| | PHE1 | TRP2 | LEU3 | GLN4 | PROS | LEU6 | ALA7 | ASP8 | ALA9 |
|---|---|---|---|---|---|---|---|---|---|
| ALA | −9.7 | −10.0 | −9.8 | −10.1 | | −10.0 | | −10.4 | |
| CYS | | −10.0 | −9.8 | −9.9 | | | | −9.7 | −9.8 |
| ASP | | | −10.3 | −10.8 | −9.7 | −10.1 | | | |
| GLU | | | −9.9 | −10.1 | | −10.8 | −10.1 | −9.9 | −9.7 |
| PHE | | −10.1 | −10.2 | −10.0 | | −10.5 | −10.2 | −10.1 | −10.4 |
| GLY | | −10.1 | | −9.7 | | −10.2 | −9.9 | | |
| HIS | | | −9.7 | −9.8 | −9.7 | −9.7 | | −9.8 | −10.1 |
| ILE | −9.7 | −10.1 | −10.0 | −9.8 | | −9.9 | | | |
| LYS | | | | −9.7 | | | | −9.9 | |
| LEU | | −10.1 | | −10.1 | | | −9.8 | | −10.3 |
| MET | | | | | | | −9.7 | | −9.7 |
| ASN | | | −10.4 | −10.2 | | −10.6 | | | −9.9 |
| PRO | | | −10.2 | −10.8 | | −10.2 | −9.8 | −10.3 | −10.2 |
| GLN | | −9.8 | −10.2 | | | −10.0 | −10.0 | −10.0 | |
| ARG | | −9.9 | −9.9 | | | | | −9.9 | |
| SER | | −10.0 | −9.7 | −10.2 | | | | −10.1 | −10.0 |
| THR | | −9.9 | −9.8 | −9.8 | | −10.2 | | −10.5 | |
| VAL | | −9.8 | −10.1 | | | −9.9 | −10.0 | −9.8 | −10.1 |
| TRP | −10.8 | | −10.3 | −9.9 | | | −10.5 | −10.4 | −10.3 |
| TYR | −10.0 | −10.2 | −9.8 | | | −10.4 | −10.2 | −10.7 | |

FWLQPLADA and its mutations at the corresponding position:
1: W, Y, A, or I
2: Y, F, G, I, L, A, C, S, R, T, Q, or V
3: N, D, W, F, P, Q, V, I, E, R, A, C, T, Y, H, or S
4: D, P, N, S, A, E, L, F, C, W, H, I, T, G, or K
5: D, or H
6: E, N, F, Y, G, P, T, D, A, Q, I, V, or H
7: W, F, Y, E, Q, V, G, L, P, or M
8: Y, T, A, W, P, F, S, Q, E, K, R, H, V, or C
9: F, L, W, P, H, V, S, N, C, E, or M

TABLE 6
Reengineering of the best epitope from HPV-18.
GGQTVQVYF peptide (WT energy: −9.2 kcal/mol)

| | GLY1 | GLY2 | GLN3 | THR4 | VAL5 | GLN6 | VAL7 | TYR8 | PHE9 |
|---|---|---|---|---|---|---|---|---|---|
| ALA | | −9.3 | −9.3 | −9.6 | | | −9.3 | | |
| CYS | −9.2 | | | | | | | | |
| ASP | | −9.4 | −9.2 | −9.2 | | | −9.4 | −9.6 | |
| GLU | | | | | | −9.3 | | | |
| PHE | −9.7 | −9.4 | −9.4 | −9.6 | | | | −9.3 | |
| GLY | | | −9.2 | −9.3 | −9.4 | −9.3 | | | |
| HIS | | | −9.5 | −9.3 | | | | | |
| ILE | −9.2 | | | | | | | | |
| LYS | | | | | | | | | |
| LEU | −9.2 | | −9.4 | | | | | | |
| MET | | | | | | | | | |
| ASN | | | | −9.2 | | −9.4 | | | |
| PRO | −9.6 | −9.8 | −9.4 | −9.5 | | −9.3 | | | |
| GLN | | −9.3 | | −9.2 | | | | | |
| ARG | −9.4 | | −9.5 | −9.3 | | | | | |
| SER | | −9.2 | −9.2 | −9.2 | | | | | |
| THR | | | | | −9.3 | | −9.4 | | |
| VAL | | | −9.5 | | | | | | |
| TRP | −9.8 | | −9.8 | −9.8 | −9.6 | −9.2 | | −9.5 | −9.4 |
| TYR | −9.7 | −9.3 | −9.7 | −9.3 | | | | | |

GGQTVQVYF and its mutations at the corresponding position:
1: W, F, Y, P, R, C, I, or L
2: P, D, F, A, Q, Y, or S
3: W, Y, H, R, V, F, L, P, A, D, G, or S
4: W, A, F, P, G, H, R, Y, D, N, Q, or S
5: W, G, or T
6: N, E, G, P, or W
7: D, T, or A
8: D, W, or F
9: W

### Example 2: Identification of Epitopes from the Proteomes of HPV6 (777 Sequences)

From those sequences, a set of preliminary T cell epitopes were obtained, which were passed through multiple filtering process of discarding repetitive sequences, aligning against proteins of 148 (and/or suitable number of) species of Fusobacterium nucleatum and those belong to Lactobacillus genus. Through this process, a set of common epitopes between HPV and Lactobacillus proteins were obtained. As an example, an epitope (KNALTTAEI) common between HPV type 6 and Fusobacterium nucleatum, L. equigenerosi, L. fuchuensis, and L. odoratitofui proteins was determined. Those epitopes that did not fit with the criteria were discarded.

The list of epitopes can additionally or alternatively include those shown in the next tables (Table 7). Additionally, the energy of binding from docking simulations, expressed in kcal/mol, is also listed. The energy of binding can be understood as a ranking to determine epitopes with a higher affinity (lower energy of binding) for the HLA class I receptor.

TABLE 7
List of de novo epitopes suggested for
prevention of warts, such as genital warts,
and/or papillomas (HPV type 6).
A) Filter 1: Predicted HPV epitopes found in
bacteria with more than 70% of
sequence identity and 100% matches.
PREVENTION OF WARTS AND/OR PAPILLOMAS
HPV6

| | Energy of | |
|---|---|---|
| | binding | |
| Epitope | (kcal/mol) | Lactobacillus sp. |
| IGARIHYFY | −9.8 | oryzae (IGAAIGYFY), |
| | | malefermentans |
| | | (IGAAIGYFY) |
| GTAGILELL | −8.1 | sp-wkB8 (GTAGILELL) |

B) Filter 2: Predicted HPV epitopes
found in bacteria with 100% of
sequence identity and 8 matches.
PREVENTION OF WARTS AND/OR PAPILLOMAS
HPV6

| | Energy of | |
|---|---|---|
| | binding | |
| Epitope | (kcal/mol) | Lactobacillus sp. |
| LVLTLLLYL | −8.5 | capillatus, apodemi, |
| | | dextrinicus |
| SVLVLTLLL | −7.8 | coryniformis |
| | | fuchuensis, apodemi, |
| | | camelliae, composti, |
| | | thailandensis, vini |

C) Filter 3: Predicted HPV epitopes
found in bacteria with 100% of
sequence identity and 7 matches.
PREVENTION OF WARTS AND/OR PAPILLOMAS
HPV6

| | Energy of | |
|---|---|---|
| | binding | |
| Epitope | (kcal/mol) | Lactobacillus sp. |
| DPYKNLSFW | −9.6 | wasatchensis |
| VVLPDPNKF | −9.2 | johnsonii |
| KHAIVTVTY | −9.1 | fabifermentans |
| NTMDYVVW | −9.1 | brevis |
| T | | |
| NVVKIPPTI | −9 | plantarum |
| YPDYLQMAA | −9 | agilis |
| PVFITGSGF | −9 | ruminis |
| PTQHPVTNI | −8.8 | sanfranciscensis, backii, |
| | | florum, lindneri, oeni |
| LLLTTPLQF | −8.7 | rennini |
| VVCFVSIIL | −8.7 | agilis, ruminis |
| CAFIVGVLG | −8.6 | collinoides |
| ISFLGGTVI | −8.5 | vini |
| KAKQMGLSH | −8.4 | diolivorans, parafarraginis |
| TSSETTTPA | −8.4 | backii |
| APTITSHPI | −8.4 | koreensis |
| PVQIAAGTT | −8.3 | ingluviei |
| HPYFSIKRA | −8.3 | florum |
| NSHLATPCV | −8.2 | kimchicus |
| VLGLLLMHY | −8.2 | diolivorans, florum, rennini, |
| | | rossiae |
| TVVPKVSGY | −8.1 | nodensis |
| RTGISNAST | −8.1 | sharpeae |
| VQIAAGTTS | −8 | ingluviei |
| KNALTTAEI | −8 | equigenerosi, fuchuensis, |
| | | odoratitofui |
| VARTLATLL | −8 | plantarum, senioris |
| PAVSKASAA | −7.9 | bifermentans, coryniformis, |
| | | fermentum, rennini, saniviri, |
| | | tucceti |
| KQDILDVLI | −7.9 | diolivorans |
| TVQDLKRKY | −7.9 | plantarum |
| TVARTLATL | −7.9 | plantarum |
| QIAAGTTST | −7.8 | parabrevis |
| NGKYVMAA | −7.8 | acetotolerans |
| Q | | |
| DSNVRLVVQ | −7.8 | lindneri |
| HCYEQLVDS | −7.8 | fermentum |
| MEAIAKRLD | −7.7 | ahmentarius, coleohominis, |
| | | coleohominis-DSH, |
| | | concavus, coryniformis, |
| | | diolivorans, farraginis, |
| | | fermentum, frumenti, |
| | | manihotivorans, |
| | | oligofermentans, oris- |
| | | PB013-T2-3, oryzae, rennini, |
| | | secaliphilus, suebicus, |
| | | vaccinostercus, viridescens, |
| | | wasatchensis, zymae |
| PNNGKYVMA | −7.7 | acetotolerans |
| NNGKYVMA | −7.7 | acetotolerans |
| A | | |
| TVETTTSSL | −7.6 | vaccinostercus |
| MESANASTS | −7.5 | oryzae |
| IPPTIRHKL | −7.5 | mellis |
| RKHKALTLI | −7.2 | cacaonum, frumenti, mali |
| SRARRRKRA | −6.7 | olsenella-uli |

Epitopes are classified according to docking energy of binding expressed in kcal/mol. Additionally, the column of Lactobacillus sp totocontains the species where the epitope was found.

In a next stage (and/or at any suitable time and frequency), in a variation, to improve the affinity of epitopes for the receptor, it is also possible to subject the best epitopes to a reengineering, which means that every amino acid can be mutated in-silico, one at the time, by the other 19 amino acids. Those new epitopes obtained by reengineering can be tested by docking and/or other suitable techniques, and then classified according their energy of binding to the receptor. In this way, it is possible to obtain new epitopes with a better affinity to the receptor.

Thus, as another example, a preferred set of epitopes (e.g., with best docking energy score), obtained from HPV type 6 were selected for reengineering. In addition, we can include information in Table 8. For each table, the first row represent the amino acid composition of each one of those WT epitopes. Next rows are representing the 20 amino acids to which each position was mutated, and the respective docking energy values, being represented only those having equal or higher affinity (e.g lower energy) than obtained for each WT epitope (showed in the table header). Below Table 8, mutations improving the affinity of each epitope are disclosed.

TABLE 8
Reengineering of the best epitope from HPV-6.
IGARIHYFY peptide (WT energy: −9.8 kcal/mol)

| | ILE1 | GLY2 | ALA3 | ARG4 | ILE5 | HIS6 | TYR7 | PHE8 | TYR9 |
|---|---|---|---|---|---|---|---|---|---|
| ALA | −10 | | | −10 | −9.8 | | | | |
| CYS | | | | | | | | | |
| ASP | | | | | −10 | | | | |
| GLU | −9.9 | | | | | | | | |
| PHE | | | | −9.8 | | | | | |
| GLY | | | | | | | | | |
| HIS | −9.8 | | | −9.8 | | | | | |
| ILE | | | | | | | | | |
| LYS | | | | | | | | | |
| LEU | | | | −10 | | | | | |
| MET | | | | | | | | | |
| ASN | | | | | −10.2 | | | | |
| PRO | | | | −9.8 | | | | −9.8 | |
| GLN | | | | | | −9.9 | | | |
| ARG | | | | | | | | | |
| SER | | | −9.9 | | | | | | |
| THR | | | | | −9.8 | | | | |
| VAL | −9.8 | | | | | | | | |
| TRP | | | | | | | | | |
| TYR | | −10.3 | | −10.2 | −9.8 | | | | |

IGARIHYFY and its mutations at the corresponding position:
1: A, E, H, or V
2: Y
3: S
4: Y, A, L, F, H, or P
5: N, D, A, T, or Y
6: Q
8: P

### Example 3: Identification of Epitopes from the Proteomes of HPV11 (719 Sequences)

From those sequences, a set of preliminary T cell epitopes were obtained, which were passed through multiple filtering process of discarding repetitive sequences, aligning against proteins of 148 (and/or suitable number of) species of Fusobacterium nucleatum and those belong to Lactobacillus genus. Through this process, a set of common epitopes were obtained between HPV and Lactobacillus proteins, including one epitope (QIAAATTTT) common between HPV (e.g., HPV type 11) and L. iners and L. crispatus proteins. Those epitopes that did not fit with the criteria were discarded.

As an example of use, those 46 (and/or suitable number) peptide sequences as epitopes against HPV infection to warts, which represents to 65 (and/or suitable number) Lactobacillus species. The list of epitopes can additionally or alternatively include those shown in the next tables (Table 9). Additionally, the energy of binding from docking simulations, expressed in kcal/mol, is also listed. The energy of binding can be understood as a ranking to determine epitopes with a higher affinity (lower energy of binding) for the HLA class I receptor.

TABLE 9
List of de novo epitopes suggested for prevention of warts, such as
genital warts, and/or papillomas (HPV type 11), predicted HPV epitopes
found in bacteria with 100% of sequence identity and 7 matches.
PREVENTION OF WARTS AND/OR PAPILLOMAS

| | Energy of | |
|---|---|---|
| | binding | |
| Epitope | (kcal/mol) | Lactobacillus sp. |
| PVFITGSDF | −9.5 | hokkaidonensis, plantarum, tucceti |
| LSTPQRLVT | −9.4 | perolens |
| FVVAVLGLL | −9.4 | malefermentans, senmaizukei |
| TPFSPVTPA | −9.2 | similis |
| LPVVIAFAV | −9.1 | backii |
| LVAAENDTF | −9.1 | parafarraginis |
| PSDSTVYVP | −8.9 | floricola |
| GAPEVVPPT | −8.9 | harbinensis, viridescens |
| SDSTVYVPP | −8.8 | floricola |
| QGNTVEVKF | −8.8 | vini |
| LVLTLLLYL | −8.7 | capillatus, apodemi, dexfrinicus |
| RVGLYSRAL | −8.6 | equi |
| LILPVVIAF | −8.5 | lindneri |
| TSSESTTPA | −8.4 | algidus, apodemi |
| DSNVRLVVE | −8.4 | lindneri |
| VLIILISDF | −8.4 | amylophilus, hokkaidonensis, kimchicus, |
| | | odoratitofui, rhamnosus |
| KPRARRRKR | −8.3 | olsenella-uli |
| VQIAAATTT | −8.3 | ghanensis, nodensis |
| RRLFETREL | −8.3 | selangorensis |
| LTDAKVALL | −8.3 | fabifermentans, similis |
| MADDSALYE | −8.2 | zymae |
| EVVPVQIAA | −8.2 | plantarum, ruminis |
| NAVYELSDA | −8.2 | coryniformis |
| SSESTTPAI | −8.2 | mellifer |
| QIAAATTTT | −8.1 | acetotolerans, acidophilus, amylolyticus, brantae, |
| | | coleohominis, concavus, crispatus, delbrueckii, |
| | | dextrinicus, hamsteri, helveticus, hominis, iners, |
| | | jensenii, johnsonii, kalixensis, kefiranofaciens, |
| | | kunkeei, mellifer, nasuensis, nodensis, pasteurii, |
| | | sakei, saniviri, selangorensis, sp-ASF360, sp-wkB8, |
| | | xiangfangensis |
| KIQSGVRAL | −8.1 | kisonensis |
| TVARTLGTL | −8.1 | acidophilus, crispatus |
| STSATSIDQ | −8 | fabifermentans |
| TSSLTITTS | −8 | florum |
| ETNEDILKV | −8 | nodensis, tucceti |
| TVQSTTSSL | −7.9 | odoratitofui |
| RALQQVQVT | −7.9 | fabifermentans |
| LKDIVLDLQ | −7.8 | senmaizukei |
| PVQIAAATT | −7.8 | nodensis |
| YSIKKVINKT | −7.8 | apodemi |
| PRARRRKRA | −7.7 | olsenella-uli |
| ASTSATSID | −7.6 | fabifermentans |
| RKHRALTLI | −7.5 | sharpeae |
| ADDSALYEK | −7.3 | zymae |
| KCKDIRSTL | −7.3 | jensenii |
| STTSSLTIT | −7.2 | cacaonum |
| RKTACRRRL | −7.1 | aviarius |
| VVIAFAVCI | −7 | backii, phage-phiAQ113 |
| AIAKRLDAC | −7 | alimentarius, helveticus, rossiae |
| MEVVPVQIA | −7 | plantarum, ruminis |
| TAPTVSACT | −6.8 | frumenti |

Epitopes are classified according to docking energy of binding expressed in kcal/mol. Additionally, the column of Lactobacillus sp.contains the species where the epitope was found.

In a next stage (and/or at any suitable time and frequency), in a variation, to improve the affinity of epitopes for the receptor, it is also possible to subject the best epitopes to a reengineering, which means that every amino acid can be mutated in-silico, one at the time, by the other 19 amino acids. Those new epitopes obtained by reengineering can be tested by docking and/or other suitable techniques, and then classified according their energy of binding to the receptor. In this way, it is possible to obtain new epitopes with a better affinity to the receptor.

Thus, as another example, a preferred set of epitopes (e.g., with best docking energy score), obtained from HPV type 11 was selected for reengineering, as shown in Table 10. The first row represents the amino acid composition of each one of those wild-type (WT) epitopes. Next rows are representing the 20 amino acids to which each position was mutated, and the respective docking energy values, being represented only those having equal or higher affinity (e.g lower energy) than obtained for each WT epitope (showed in the table header). Below Table 10, mutations improving the affinity of each epitope are disclosed.

TABLE 10
Reengineering of the best epitope from HPV type 11, showing
energy only of the substitutions best
PVFITGSDF peptide (WT energy: −9.5 kcal/mol)

| | PRO1 | VAL2 | PHE3 | ILE4 | THR5 | GLY6 | SER7 | ASP8 | PHE9 |
|---|---|---|---|---|---|---|---|---|---|
| ALA | | | | | | −9.6 | | | |
| CYS | | | | | | −9.5 | | | |
| ASP | | −9.5 | | | | | | | |
| GLU | | | | | | | | | |
| PHE | −9.6 | −9.6 | | | | | −9.6 | | |
| GLY | | | | | | | | | |
| HIS | | | | | | | | | |
| ILE | | | | | | | | | |
| LYS | | | | | | | | | |
| LEU | | | | | | | | | |
| MET | | | | | | | | | |
| ASN | | | | | | | | | |
| PRO | | | | | −10.0 | −9.8 | −9.5 | | |
| GLN | | | | | | | −9.7 | | |
| ARG | | | | | | | | | |
| SER | | | | | | | | | |
| THR | | | | | | | | | |
| VAL | | | | | | | | | |
| TRP | −9.9 | −9.5 | | | | | −9.9 | −9.7 | −9.9 |
| TYR | | | | | | | | | |

PVFITGSDF and its mutations at the corresponding position:
1: W, or F
2: F, D, or W
5: P
6: P, A, or C
7: W, Q, F, or P
8: W
9: W

### Example 4: Identification of Epitopes from the Proteomes of HPV16

As an example of an embodiment of a method (e.g. portions described herein), 1278 proteomic sequences of HPV type 16 were identified.

From those sequences, a set of preliminary T cell epitopes were obtained, which were passed through multiple filtering process of discarding repetitive sequences, aligning against proteins of 148 (and/or suitable number of) species of Fusobacterium nucleatum and those belong to Lactobacillus genus. Through this process, a set of common epitopes were obtained between HPV and Lactobacillus proteins, including one epitope (RAAKRRLFE) common between HPV (e.g., HPV type 16) and L. crispatus proteins. Those epitopes that did not fit with the criteria were discarded.

As an example of use, those 52 (and/or suitable number) peptide sequences as epitopes against HPV infection to cancer, represent to 48 (and/or suitable number) Lactobacillus species. The list of epitopes can additionally or alternatively include those shown in the next tables (Table 11). Additionally, the energy of binding from docking simulations, expressed in kcal/mol, is also listed. The energy of binding can be understood as a ranking to determine epitopes with a higher affinity (lower energy of binding) for the HLA class I receptor.

TABLE 11
List of de novo epitopes suggested for prevention of cancer, such as
cervical cancer (HPV type 16).
A) Filter 1: Predicted HPV type 16 epitopes found in bacteria with
more than 70% of sequence identity and 100% matches.
PREVENTION OF CANCER

| | Energy of binding | |
|---|---|---|
| Epitope | (kcal/mol) | Lactobacillus sp. |
| LLKLLGSTW | −8.5 | Acidophilus (LLKLVGSTS), floricola |
| | | (LLTLLGSPW), nasuensis |
| | | (LGKWLGSTW), plantarum |
| | | (LLMLLGLTW), sakei |
| | | (LAKLLGSGW) |

B) Filter 2: Predicted HPV16 epitopes found in bacteria with 100% of
sequence identity and 8 matches.
PREVENTION OF CANCER

| | Energy of binding | |
|---|---|---|
| Epitope | (kcal/mol) | Lactobacillus sp. |
| SAFLKSNSQ | −8.1 | tucceti |
| PPTPAETGG | −8 | parafarraginis |
| VEKKTGDAI | −7.4 | casei |

C) Filter 3: Predicted HPV16 epitopes found in bacteria with 100% of
sequence identity and 7 matches.
PREVENTION OF CANCER

| | Energy of binding | |
|---|---|---|
| Epitope | (kcal/mol) | Lactobacillus sp. |
| FWLQPLADA | −9.7 | mucosae-LM1, harbinensis, kisonensis, |
| | | shenzhenensis, suebicus, |
| | | malefermentans |
| VAVNPGDCP | −9.4 | farraginis, spicheri, brevis, senmaizukeicasei |
| FELSQMVQW | −9.2 | buchneri |
| ADPAAATKY | −9.2 | koreensis |
| HAASPTSIN | −9.2 | hominis |
| LWLPSEATV | −9 | xiangfangensis |
| DPAGTNGEE | −9 | rossiae |
| DAGDFYLHP | −9 | agilis |
| PFDENGNPV | −8.9 | tucceti, mucosae-LM1, hamsteri, jensenii, |
| | | odoratitofui, sp-wkB8 |
| INHQVVPTL | −8.8 | harbinensis |
| LADTNSNAS | −8.8 | mucosae-LM1 |
| QPPTPAETG | −8.7 | parafarraginis |
| SHAASPTSI | −8.7 | hominis |
| IAEQILQYG | −8.7 | algidus |
| YDLSTIDPA | −8.6 | gastricus |
| YSLYGTTLE | −8.5 | siliginis |
| IPIVPGSPQ | −8.5 | wasatchensis |
| LAKFKELYG | −8.5 | amylophilus |
| YGNTEVETQ | −8.5 | bifermentans |
| LGIGTGSGT | −8.4 | collinoides, spicheri, diolivorans, koreensis, zymae, |
| | | brevis, similis, parabrevis |
| TPSIADSIK | −8.3 | spicheri, koreensis |
| DYLTQAETE | −8.1 | ruminis |
| SGKSIGAKV | −8.1 | acetotolerans |
| TPAILDINN | −8.1 | suebicus |
| GGLGIGTGS | −8.1 | spicheri, wasatchensis, koreensis, zymae, brevis, |
| | | parabrevis |
| AGTSRLLAV | −8 | fabifermentans |
| LGKRKATPT | −8 | malefermentans |
| MADPAAATK | −8 | koreensis |
| IGNKQTLRT | −8 | malefermentans |
| TPPRPIPKP | −7.9 | olsenella-uli |
| ATKYPLLKL | −7.9 | curieae |
| ICEEASVTV | −7.9 | jensenii |
| SLIPIVPGS | −7.9 | wasatchensis |
| PYLHNRLVV | −7.9 | buchneri |
| AEEIELQTI | −7.8 | ginsenosidimutans |
| TAHALFTAQ | −7.8 | zymae, capillatus |
| NASAFLKSN | −7.8 | tucceti |
| CEEASVTVV | −7.7 | jensenii |
| KPYWLQRAQ | −7.6 | brevis |
| GNQLFVTVV | −7.6 | saniviri |
| AAMLAKFKE | −7.6 | sharpeae |
| GEDLVDFIV | −7.6 | parafarraginis, farraginis, brevis-subsp- |
| | | gravesensis |
| LYLHIQSLA | −7.5 | rossiae |
| RAAKRRLFE | −7.5 | crispatus |
| AHTKDGLTV | −7.4 | kunkeei, xiangfangensis, composti |
| NALDGNLVS | −7.4 | curieae, delbrueckii |
| VPTLAVSKN | −7.2 | sharpeae |
| TLCQRLNVC | −7.2 | koreensis |

Epitopes are classified according to docking energy of binding expressed in kcal/mol. Additionally, the column of Lactobacillus sp. contains the species where the epitope was found.

In a next stage (and/or at any suitable time and frequency), in a variation, to improve the affinity of epitopes for the receptor, it is also possible to subject the best epitopes to a reengineering, which means that every amino acid can be mutated in-silico, one at the time, by the other 19 amino acids. Those new epitopes obtained by reengineering can be tested by docking and/or other suitable techniques, and then classified according their energy of binding to the receptor. In this way, it is possible to obtain new epitopes with a better affinity to the receptor.

Thus, as another example, a preferred set of epitopes (e.g., with best docking energy score), obtained from HPV type 16 was selected for reengineering, as shown in Table 12. The first row represents the amino acid composition of each one of those wild-type (WT) epitopes. Next rows are representing the 20 amino acids to which each position was mutated, and the respective docking energy values, being represented only those having equal or higher affinity (e.g lower energy) than obtained for each WT epitope (showed in the table header). Below Table 12, mutations improving the affinity of each epitope are disclosed.

TABLE 12
Reengineering of the best epitope from HPV type 16, showing
energy only of the best substitutions
FWLQPLADA peptide (WT energy −9.7 kcal/mol)

| | PHE1 | TRP2 | LEU3 | GLN4 | PRO5 | LEU6 | ALA7 | ASP8 | ALA9 |
|---|---|---|---|---|---|---|---|---|---|
| ALA | −9.7 | −10.0 | −9.8 | −10.1 | | −10.0 | | −10.4 | |
| CYS | | −10.0 | −9.8 | −9.9 | | | | −9.7 | −9.8 |
| ASP | | | −10.3 | −10.8 | −9.7 | −10.1 | | | |
| GLU | | | −9.9 | −10.1 | | −10.8 | −10.1 | −9.9 | −9.7 |
| PHE | | −10.1 | −10.2 | −10.0 | | −10.5 | −10.2 | −10.1 | −10.4 |
| GLY | | −10.1 | | −9.7 | | −10.2 | −9.9 | | |
| HIS | | | −9.7 | −9.8 | −9.7 | −9.7 | | −9.8 | −10.1 |
| ILE | −9.7 | −10.1 | −10.0 | −9.8 | | −9.9 | | | |
| LYS | | | | −9.7 | | | | −9.9 | |
| LEU | | −10.1 | | −10.1 | | | −9.8 | | −10.3 |
| MET | | | | | | | −9.7 | | −9.7 |
| ASN | | | −10.4 | −10.2 | | −10.6 | | | −9.9 |
| PRO | | | −10.2 | −10.8 | | −10.2 | −9.8 | −10.3 | −10.2 |
| GLN | | −9.8 | −10.2 | | | −10.0 | −10.0 | −10.0 | |
| ARG | | −9.9 | −9.9 | | | | | −9.9 | |
| SER | | −10.0 | −9.7 | −10.2 | | | | −10.1 | −10.0 |
| THR | | −9.9 | −9.8 | −9.8 | | −10.2 | | −10.5 | |
| VAL | | −9.8 | −10.1 | | | −9.9 | −10.0 | −9.8 | −10.1 |
| TRP | −10.8 | | −10.3 | −9.9 | | | −10.5 | −10.4 | −10.3 |
| TYR | −10.0 | −10.2 | −9.8 | | | −10.4 | −10.2 | −10.7 | |

FWLQPLADA and its mutations at the corresponding position:
1: W, Y, A, or I
2: Y, F, G, I, L, A, C, S, R, T, Q, or V
3: N, D, W, F, P, Q, V, I, E, R, A, C, T, Y, H, or S
4: D, P, N, S, A, E, L, F, C, W, H, I, T, G, or K
5: D, or H
6: E, N, F, Y, G, P, T, D, A, Q, I, V, or H
7: W, F, Y, E, Q, V, G, L, P, or M
8: Y, T, A, W, P, F, S, Q, E, K, R, H, V, or C
9: F, L, W, P, H, V, S, N, C, E, or M

### Example 5: Identification of Epitopes from the Proteomes of HPV18

As an example of an embodiment of a method (e.g. portions described herein), epitopes from 740 proteomic sequences of HPV type 18 were identified.

From those sequences, a set of preliminary T cell epitopes were obtained, which were passed through multiple filtering process of discarding repetitive sequences, aligning against proteins of 148 (and/or suitable number of) species of Fusobacterium nucleatum and those belong to Lactobacillus genus. Through this process, a set of common epitopes were obtained between HPV and Lactobacillus proteins, including two epitopes (KGGQTVQVY and GGQTVQVYF) common between HPV (e.g., HPV type 18) and L. crispatus proteins. Those epitopes that did not fit with the criteria were discarded.

As an example, 13 (and/or suitable number) peptide sequences as epitopes against HPV infection to cancer were used, which represents to 12 (and/or suitable number) Lactobacillus species. The list of epitopes can additionally or alternatively include those shown in the next tables (Table 13). Additionally, the energy of binding from docking simulations, expressed in kcal/mol, is also listed. The energy of binding can be understood as a ranking to determine epitopes with a higher affinity (lower energy of binding) for the HLA class I receptor.

TABLE 13
List of de novo epitopes suggested for prevention
of cancer, such as cervical cancer (HPV type 18).
A) Filter 1: Predicted HPV18 epitopes found in
bacteria with more than 70% of sequence
identity and 100% matches.
PREVENTION OF CANCER

| | Energy of | |
|---|---|---|
| Epitope | (kcal/mol) | Lactobacillus sp. |
| QTQLCILGC | −7.5 | Floricola (QKQLEILGC) |

B) Filter 2: Predicted HPV18 epitopes found in
bacteria with 100% of sequence identity and 8
matches.
PREVENTION OF CANCER

| | Energy of | |
|---|---|---|
| | binding | |
| Epitope | (kcal/mol) | Lactobacillus sp. |
| GGQTVQVYF | −9.2 | crispatus |
| QATTKDGNS | −7.8 | similis |

C) Filter 3: Predicted HPV18 epitopes found in
bacteria with 100% of sequence identity and 7
matches.
PREVENTION OF CANCER

| | Energy of | |
|---|---|---|
| | binding | |
| Epitope | (kcal/mol) | Lactobacillus sp. |
| KNGNPVYEI | −9.1 | suebicus |
| KGGQTVQVY | −8.7 | crispatus |
| ICGHYIILF | −8.7 | oris-PB013-T2-3 |
| KQGAMLAVF | −8.6 | johnsonii |
| SIVDLSTHF | −8.4 | perolens |
| HRFSTSDDT | −8.2 | bifermentans |
| KSRLTVAKG | −8.1 | collinoides |
| QHRFSTSDD | −8.1 | bifermentans |
| KAHKAIELQ | −7.6 | plantarum |
| ETLSERLSC | −5.9 | brevis-subsp-gravesensis |

Epitopes are classified according to docking energy of binding expressed in kcal/mol. Additionally, the column of Lactobacillus sp. contains the species where the epitope was found.

In a next stage (and/or at any suitable time and frequency), in a variation, to improve the affinity of epitopes for the receptor, it is also possible to subject the best epitopes to a reengineering, which means that every amino acid can be mutated in-silico, one at the time, by the other 19 amino acids. Those new epitopes obtained by reengineering can be tested by docking and/or other suitable techniques, and then classified according their energy of binding to the receptor. In this way, it is possible to obtain new epitopes with a better affinity to the receptor.

Thus, as another example, a preferred set of epitopes (e.g., with best docking energy score), obtained from HPV type 18 was selected for reengineering, as shown in Table 14. The first row represents the amino acid composition of each one of those wild-type (WT) epitopes. Next rows are representing the 20 amino acids to which each position was mutated, and the respective docking energy values, being represented only those having equal or higher affinity (e.g lower energy) than obtained for each WT epitope (showed in the table header). Below Table 14, single mutations improving the affinity of each epitope are disclosed.

TABLE 14
Reengineering of the best epitope from HPV type 18, showing
energy only of the best substitutions
GGQTVQVYF peptide (WT energy: −9.2 kcal/mol)

| | GLY1 | GLY2 | GLN3 | THR4 | VAL5 | GLN6 | VAL7 | TYR8 | PHE9 |
|---|---|---|---|---|---|---|---|---|---|
| ALA | | −9.3 | −9.3 | −9.6 | | | −9.3 | | |
| CYS | −9.2 | | | | | | | | |
| ASP | | −9.4 | −9.2 | −9.2 | | | −9.4 | −9.6 | |
| GLU | | | | | | −9.3 | | | |
| PHE | −9.7 | −9.4 | −9.4 | −9.6 | −9.3 | | | | |
| GLY | | | −9.2 | −9.3 | −9.4 | −9.3 | | | |
| HIS | | | −9.5 | −9.3 | | | | | |
| ILE | −9.2 | | | | | | | | |
| LYS | | | | | | | | | |
| LEU | −9.2 | | −9.4 | | | | | | |
| MET | | | | | | | | | |
| ASN | | | | −9.2 | | −9.4 | | | |
| PRO | −9.6 | −9.8 | −9.4 | −9.5 | | −9.3 | | | |
| GLN | | −9.3 | | −9.2 | | | | | |
| ARG | −9.4 | | −9.5 | −9.3 | | | | | |
| SER | | −9.2 | −9.2 | −9.2 | | | | | |
| THR | | | | | −9.3 | | −9.4 | | |
| VAL | | | −9.5 | | | | | | |
| TRP | −9.8 | | −9.8 | −9.8 | −9.6 | −9.2 | | −9.5 | −9.4 |
| TYR | −9.7 | −9.3 | −9.7 | −9.3 | | | | | |

GGQTVQVYF and its mutations at the corresponding position:
1: W, F, Y, P, R, C, I, or L
2: P, D, F, A, Q, Y, or S
3: W, Y, H, R, V, F, L, P, A, D, G, or S
4: W, A, F, P, G, H, R, Y, D, N, Q, or S
5: W, G, or T
6: N, E, G, P, or W
7: D, T, or A
8: D, W, or F
9: W

### Example 6: Antibody-DNA Conjugates

The concept of DNA labeled binding proteins was put to a test using two different antibodies:
Anti-mouse IgG antibody (goat origin), which binds specifically to mouse IgG protein
Anti-C. difficile Toxin B (sheep origin), which binds specifically to Toxin B of Clostridium difficile
Anti-C. difficile Toxin B (mouse origin), which binds specifically to Toxin B of Clostridium difficile

### Results for Binding Protein-DNA Conjugation:

Successful conjugation of THIOL (as R group) modified oligonucleotides to three antibodies against different targets (Sheep polyclonal anti-Toxin B C. difficile, mouse monoclonal anti-Toxin B C. difficile, goat anti-mouse IgG) demonstrated by the shift band to a higher molecular weight compared to unconjugated antibodies+free oligos. Characterization of oligo antibodies showed that oligos were attached to both heavy and light chains.

### Results for Target Binding, Amplification and Detection of Targets:

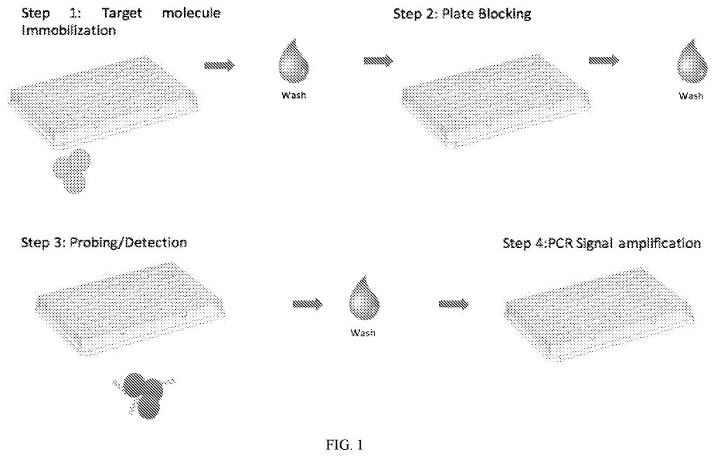

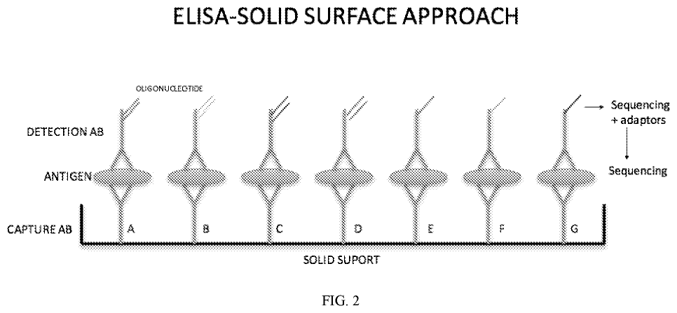

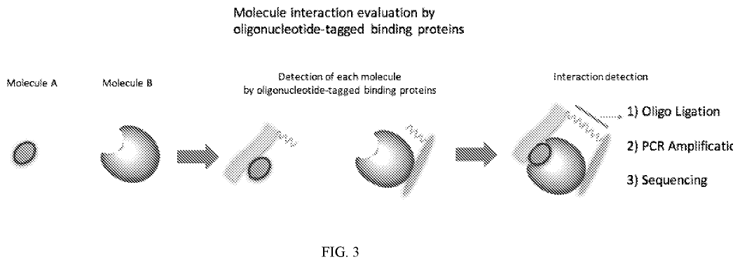

The performance of the obtained antibody-DNA conjugates were evaluated by PCR (FIG. 1) or detection of their respective protein target (for example, Toxin B C. difficile and mouse IgG). Positive signals by conventional PCR were obtained for all the antibody-DNA conjugates tested. Also, no-protein target controls containing only coating buffer were included in each assay to measure the background signal. Different strategies of detection were tested (FIG. 2) and evaluation of more targets on the same reaction (FIG. 3)

1. A method for detecting a target molecule, the method comprising the steps of:
a) Conjugating of a DNA oligonucleotide to an antibody to the target molecule to form an antibody-DNA oligonucleotide conjugate;
b) immobilizing the target molecule;
c) binding the target molecule with the antibody-DNA oligonucleotide conjugate;
d) amplifying and detecting the DNA sequence in the antibody-DNA oligonucleotide conjugate;
wherein the DNA oligonucleotide comprises one of the following structures:
5′ R-SPACER-UPST. ADAPTER-DEFINED IDENTIFIER SEQUENCE-DWNST. ADAPTER-3′, or
5′ R-SPACER-UPST. ADAPTER-DEFINED IDENTIFIER SEQUENCE-BRIDGE_LEFT 3′, or
5′ BRIDGE_RIGHT-DEFINED IDENTIFIER SEQUENCE-DWNST. ADAPTER-SPACER-R-3′,
wherein R is a reactive group selected from the group consisting of thiol, azide, NHS-ester, amine, aldehyde, hydrazine, hexynyl, octadiynyl dU, acrydite, and sulphydryl.

2. The method of claim 1, wherein a cross-linker reagent and a reducing agent are used in the conjugation step a).

3. The method of claim 2, wherein the cross-linker reagent is SMCC or sulfo-SMCC.

4. The method of claim 1, wherein the conjugating step proceeds in parallel forming multiple antibody-DNA oligonucleotide conjugates with a different DEFINED IDENTIFIER SEQUENCE tagged to each different antibody-DNA oligonucleotide conjugate.

5. The method of claim 2, wherein the reducing agent is DTT or β-mercaptoethanol.

6. The method of claim 1, wherein the amplification is performed by polymerase chain reaction (PCR) amplification using the following DNA oligonucleotide primers:
5′-SEQUENCING ADAPTER-SEQUENCING INDEX-UPST.ADAPTER-3′, and 5′-SEQUENCING ADAPTER-SEQUENCING INDEX-DWNST.ADAPTER-3′.

7. (canceled)

8. A polypeptide comprising a sequence having at least 80% identity to a sequence of X₁X₂X₃X₄X₅X₆X₇X₈X₉, wherein:
X₁ is I, A, E, H, or V;
X₂ is G or Y;
X₃ is A or S;
X₄ is R, Y, A, L, F, H, or P;
X₅ is I, N, D, A, T, or Y;
X₆ is H or O;
X₇ is Y;
X₈ is F or P;
X₉ is Y.

9. A polypeptide of claim 8, wherein the polypeptide has the sequence of IGARIHYFY.

10. (canceled)

11. (canceled)

12. A method for treating or preventing HPV6 infection comprising administering to a patient in need thereof a pharmaceutical composition comprising the polypeptide of claim 8.

13. (canceled)

14. A polypeptide comprising a sequence having at least 80% identity to a sequence of X₁₀X₁₁X₁₂X₁₃X₁₄X₁₅X₁₆X₁₇X₁₈, wherein:
X₁₀ is P, W, or F;
X₁₁ is V, F, D, or W;
X₁₂ is F;
X₁₃ is I;
X₁₄ is T or P;
X₁₅ is G P, A, or C;
X₁₆ is S, W, Q, F, or P;
X₁₇ is D or W;
X₁₈ is For W,
or a sequence having at least 80% identity to a sequence selected from the group consisting of QKQLEILGC, GGQTVQVYF, QATTKDGNS, KNGNPVYEI, HRFSTSDDT, KGGQTVQVY, KSRLTVAKG, ICGHYIILF, QHRFSTSDD, KQGAMLAVF, KAHKAIELQ, SIVDLSTHF, and ETLSERLSC,
or a sequence having at least 80% identity to a sequence of X₂₈X₂₉X₃₀X₃₁X₃₂X₃₃X₃₄X₃₅X₃₆, wherein:
X₂₈ is G, W, F, Y, P, R, C, I or L;
X₂₉ is G, P, D, F, A, Q, Y or S;
X₃₀ is Q, W, Y, H, R, V, F, L, P, A, D, G or S;
X₃₁ is T, W, A, F, P, G, H, R, Y, D, N, Q or S;
X₃₂ is V, W, G or T;
X₃₃ is Q, N, E, G, P or W;
X₃₄ is V, D, T or A;
X₃₅ is Y, D, W or F;
X₃₆ is F or W.

15. A polypeptide of claim 14; wherein the polypeptide has the sequence of PVFITGSDF.

16. A method for treating or preventing HPV11 infection comprising administering to a patient in need thereof a pharmaceutical composition comprising the polypeptide of claim 14.

17-26. (canceled)

27. A polypeptide of claim 14; wherein the polypeptide has the sequence of GGQTVQVYF.

28. A method for treating or preventing HPV18 infection comprising administering to a patient in need thereof a pharmaceutical composition comprising the polypeptide of claim 14.


SEQUENCE LISTING (167 sequences)

SEQ ID NO: 1 - protein, 9 aa, La Crosse virus
IGAAIGYFY

SEQ ID NO: 2 - protein, 9 aa, Lactobacillus sp-wkB8
GTAGILELL

SEQ ID NO: 3 - protein, 9 aa, human papillomavirus
LVLTLLLYL

SEQ ID NO: 4 - protein, 9 aa, human papillomavirus
SVLVLTLLL

SEQ ID NO: 5 - protein, 9 aa, human papillomavirus
DPYKNLSFW

SEQ ID NO: 6 - protein, 9 aa, human papillomavirus
CAFIVGVLG

SEQ ID NO: 7 - protein, 9 aa, human papillomavirus
RTGISNAST

SEQ ID NO: 8 - protein, 9 aa, human papillomavirus
DSNVRLVVQ

SEQ ID NO: 9 - protein, 9 aa, human papillomavirus
VVLPDPNKF

SEQ ID NO: 10 - protein, 9 aa, human papillomavirus
ISFLGGTVI

SEQ ID NO: 11 - protein, 9 aa, human papillomavirus
VQIAAGTTS

SEQ ID NO: 12 - protein, 9 aa, human papillomavirus
HCYEQLVDS

SEQ ID NO: 13 - protein, 9 aa, human papillomavirus
KHAIVTVTY

SEQ ID NO: 14 - protein, 9 aa, human papillomavirus
KAKQMGLSH

SEQ ID NO: 15 - protein, 9 aa, human papillomavirus
KNALTTAEI

SEQ ID NO: 16 - protein, 9 aa, human papillomavirus
MEAIAKRLD

SEQ ID NO: 17 - protein, 9 aa, human papillomavirus
NTMDYVVWT

SEQ ID NO: 18 - protein, 9 aa, human papillomavirus
TSSETTTPA

SEQ ID NO: 19 - protein, 9 aa, human papillomavirus
VARTLATLL

SEQ ID NO: 20 - protein, 9 aa, human papillomavirus
PNNGKYVMA

SEQ ID NO: 21 - protein, 9 aa, human papillomavirus
NVVKIPPTI

SEQ ID NO: 22 - protein, 9 aa, human papillomavirus
APTITSHPI

SEQ ID NO: 23 - protein, 9 aa, human papillomavirus
PAVSKASAA

SEQ ID NO: 24 - protein, 9 aa, human papillomavirus
NNGKYVMAA

SEQ ID NO: 25 - protein, 9 aa, human papillomavirus
YPDYLQMAA

SEQ ID NO: 26 - protein, 9 aa, human papillomavirus
PVQIAAGTT

SEQ ID NO: 27 - protein, 9 aa, human papillomavirus
KQDILDVLI

SEQ ID NO: 28 - protein, 9 aa, human papillomavirus
TVETTTSSL

SEQ ID NO: 29 - protein, 9 aa, human papillomavirus
PVFITGSGF

SEQ ID NO: 30 - protein, 9 aa, human papillomavirus
HPYFSIKRA

SEQ ID NO: 31 - protein, 9 aa, human papillomavirus
TVQDLKRKY

SEQ ID NO: 32 - protein, 9 aa, human papillomavirus
MESANASTS

SEQ ID NO: 33 - protein, 9 aa, human papillomavirus
PTQHPVTNI

SEQ ID NO: 34 - protein, 9 aa, human papillomavirus
NSHLATPCV

SEQ ID NO: 35 - protein, 9 aa, human papillomavirus
TVARTLATL

SEQ ID NO: 36 - protein, 9 aa, human papillomavirus
IPPTIRHKL

SEQ ID NO: 37 - protein, 9 aa, human papillomavirus
LLLTTPLQF

SEQ ID NO: 38 - protein, 9 aa, human papillomavirus
VLGLLLMHY

SEQ ID NO: 39 - protein, 9 aa, human papillomavirus
QIAAGTTST

SEQ ID NO: 40 - protein, 9 aa, human papillomavirus
RKHKALTLI

SEQ ID NO: 41 - protein, 9 aa, human papillomavirus
VVCFVSIIL

SEQ ID NO: 42 - protein, 9 aa, human papillomavirus
TVVPKVSGY

SEQ ID NO: 43 - protein, 9 aa, human papillomavirus
NGKYVMAAQ

SEQ ID NO: 44 - protein, 9 aa, human papillomavirus
SRARRRKRA

SEQ ID NO: 45 - protein, 9 aa, Artificial Sequence
  Synthetic Sequence
  MISC_FEATURE (1)..(1): X1 is I, A, E, H, or V
  MISC_FEATURE (2)..(2): X2 is G or Y
  MISC_FEATURE (3)..(3): X3 is A or S
  MISC_FEATURE (4)..(4): X4 is R, Y, A, L, F, H, or P
  MISC_FEATURE (5)..(5): X5 is I, N, D, A, T, or Y
  MISC_FEATURE (6)..(6): X6 is H or O
  MISC_FEATURE (7)..(7): X7 is Y
  MISC_FEATURE (8)..(8): X8 is F or P
  MISC_FEATURE (9)..(9): X9 is Y
XXXXXXXXX

SEQ ID NO: 46 - protein, 9 aa, Artificial Sequence
  Synthetic Sequence
  MISC_FEATURE (1)..(1): X1 is P, W or F
  MISC_FEATURE (2)..(2): X2 is V, F, D, or W
  MISC_FEATURE (3)..(3): X3 is F
  MISC_FEATURE (4)..(4): X4 is I
  MISC_FEATURE (5)..(5): X5 is T or P
  MISC_FEATURE (6)..(6): X6 is G, P, A, or C
  MISC_FEATURE (7)..(7): X7 is S, W, Q, F, or P
  MISC_FEATURE (8)..(8): X8 is D or W
  MISC_FEATURE (9)..(9): X9 is F or W
XXXXXXXXX

SEQ ID NO: 47 - protein, 9 aa, human papillomavirus
PVFITGSDF

SEQ ID NO: 48 - protein, 9 aa, human papillomavirus
LSTPQRLVT

SEQ ID NO: 49 - protein, 9 aa, human papillomavirus
FVVAVLGLL

SEQ ID NO: 50 - protein, 9 aa, human papillomavirus
TPFSPVTPA

SEQ ID NO: 51 - protein, 9 aa, human papillomavirus
LPVVIAFAV

SEQ ID NO: 52 - protein, 9 aa, human papillomavirus
LVAAENDTF

SEQ ID NO: 53 - protein, 9 aa, human papillomavirus
PSDSTVYVP

SEQ ID NO: 54 - protein, 9 aa, human papillomavirus
GAPEVVPPT

SEQ ID NO: 55 - protein, 9 aa, human papillomavirus
SDSTVYVPP

SEQ ID NO: 56 - protein, 9 aa, human papillomavirus
QGNTVEVKF

SEQ ID NO: 57 - protein, 9 aa, human papillomavirus
LVLTLLLYL

SEQ ID NO: 58 - protein, 9 aa, human papillomavirus
RVGLYSRAL

SEQ ID NO: 59 - protein, 9 aa, human papillomavirus
LILPVVIAF

SEQ ID NO: 60 - protein, 9 aa, human papillomavirus
TSSESTTPA

SEQ ID NO: 61 - protein, 9 aa, human papillomavirus
DSNVRLVVE

SEQ ID NO: 62 - protein, 9 aa, human papillomavirus
VLIILISDF

SEQ ID NO: 63 - protein, 9 aa, human papillomavirus
KPRARRRKR

SEQ ID NO: 64 - protein, 9 aa, human papillomavirus
VQIAAATTT

SEQ ID NO: 65 - protein, 9 aa, human papillomavirus
RRLFETREL

SEQ ID NO: 66 - protein, 9 aa, human papillomavirus
LTDAKVALL

SEQ ID NO: 67 - protein, 9 aa, human papillomavirus
MADDSALYE

SEQ ID NO: 68 - protein, 9 aa, human papillomavirus
EVVPVQIAA

SEQ ID NO: 69 - protein, 9 aa, human papillomavirus
NAVYELSDA

SEQ ID NO: 70 - protein, 9 aa, human papillomavirus
SSESTTPAI

SEQ ID NO: 71 - protein, 9 aa, human papillomavirus
QIAAATTTT

SEQ ID NO: 72 - protein, 9 aa, human papillomavirus
KIQSGVRAL

SEQ ID NO: 73 - protein, 9 aa, human papillomavirus
TVARTLGTL

SEQ ID NO: 74 - protein, 9 aa, human papillomavirus
STSATSIDQ

SEQ ID NO: 75 - protein, 9 aa, human papillomavirus
TSSLTITTS

SEQ ID NO: 76 - protein, 9 aa, human papillomavirus
ETNEDILKV

SEQ ID NO: 77 - protein, 9 aa, human papillomavirus
TVQSTTSSL

SEQ ID NO: 78 - protein, 9 aa, human papillomavirus
RALQQVQVT

SEQ ID NO: 79 - protein, 9 aa, human papillomavirus
LKDIVLDLQ

SEQ ID NO: 80 - protein, 9 aa, human papillomavirus
PVQIAAATT

SEQ ID NO: 81 - protein, 9 aa, human papillomavirus
YSIKKVNKT

SEQ ID NO: 82 - protein, 9 aa, human papillomavirus
PRARRRKRA

SEQ ID NO: 83 - protein, 9 aa, human papillomavirus
ASTSATSID

SEQ ID NO: 84 - protein, 9 aa, human papillomavirus
RKHRALTLI

SEQ ID NO: 85 - protein, 9 aa, human papillomavirus
ADDSALYEK

SEQ ID NO: 86 - protein, 9 aa, human papillomavirus
KCKDIRSTL

SEQ ID NO: 87 - protein, 9 aa, human papillomavirus
STTSSLTIT

SEQ ID NO: 88 - protein, 9 aa, human papillomavirus
RKTACRRRL

SEQ ID NO: 89 - protein, 9 aa, human papillomavirus
VVIAFAVCI

SEQ ID NO: 90 - protein, 9 aa, human papillomavirus
AIAKRLDAC

SEQ ID NO: 91 - protein, 9 aa, human papillomavirus
MEVVPVQIA

SEQ ID NO: 92 - protein, 9 aa, Artificial Sequence
  Synthetic Sequence
  MISC_FEATURE (1)..(1): X10 is P, W, or F
  MISC_FEATURE (2)..(2): X11 is V, F, D, or W
  MISC_FEATURE (3)..(3): X12 is F
  MISC_FEATURE (4)..(4): X13 is I
  MISC_FEATURE (5)..(5): X14 is T or P
  MISC_FEATURE (6)..(6): X15 is G, P, A, or C
  MISC_FEATURE (7)..(7): X16 is S, W, Q, F, or P
  MISC_FEATURE (8)..(8): X17 is D or W
  MISC_FEATURE (9)..(9): X18 is F or W
XXXXXXXXX

SEQ ID NO: 93 - protein, 9 aa, Lactobacillus acidophilus
LLKLVGSTS

SEQ ID NO: 94 - protein, 9 aa, Lactobacillus floricola
LLTLLGSPW

SEQ ID NO: 95 - protein, 9 aa, Lactobacillus plantarum
LLMLLGLTW

SEQ ID NO: 96 - protein, 9 aa, Lactobacillus nasuensis
LGKWLGSTW

SEQ ID NO: 97 - protein, 9 aa, Lactobacillus sakei
LAKLLGSGW

SEQ ID NO: 98 - protein, 9 aa, Human papillomavirus
SAFLKSNSQ

SEQ ID NO: 99 - protein, 9 aa, Human papillomavirus
PPTPAETGG

SEQ ID NO: 100 - protein, 9 aa, Human papillomavirus
VEKKTGDAI

SEQ ID NO: 101 - protein, 9 aa, Human papillomavirus
FWLQPLADA

SEQ ID NO: 102 - protein, 9 aa, human papillomavirus
QPPTPAETG

SEQ ID NO: 103 - protein, 9 aa, human papillomavirus
SGKSIGAKV

SEQ ID NO: 104 - protein, 9 aa, human papillomavirus
PYLHNRLVV

SEQ ID NO: 105 - protein, 9 aa, human papillomavirus
AHTKDGLTV

SEQ ID NO: 106 - protein, 9 aa, human papillomavirus
VAVNPGDCP

SEQ ID NO: 107 - protein, 9 aa, human papillomavirus
SHAASPTSI

SEQ ID NO: 108 - protein, 9 aa, human papillomavirus
TPAILDINN

SEQ ID NO: 109 - protein, 9 aa, human papillomavirus
AEEIELQTI

SEQ ID NO: 110 - protein, 9 aa, human papillomavirus
NALDGNLVS

SEQ ID NO: 111 - protein, 9 aa, human papillomavirus
FELSQMVQW

SEQ ID NO: 112 - protein, 9 aa, human papillomavirus
IAEQILQYG

SEQ ID NO: 113 - protein, 9 aa, human papillomavirus
GGLGIGTGS

SEQ ID NO: 114 - protein, 9 aa, human papillomavirus
TAHALFTAQ

SEQ ID NO: 115 - protein, 9 aa, human papillomavirus
VPTLAVSKN

SEQ ID NO: 116 - protein, 9 aa, human papillomavirus
ADPAAATKY

SEQ ID NO: 117 - protein, 9 aa, human papillomavirus
YDLSTIDPA

SEQ ID NO: 118 - protein, 9 aa, human papillomavirus
AGTSRLLAV

SEQ ID NO: 119 - protein, 9 aa, human papillomavirus
NASAFLKSN

SEQ ID NO: 120 - protein, 9 aa, human papillomavirus
TLCQRLNVC

SEQ ID NO: 121 - protein, 9 aa, human papillomavirus
HAASPTSIN

SEQ ID NO: 122 - protein, 9 aa, human papillomavirus
YSLYGTTLE

SEQ ID NO: 123 - protein, 9 aa, human papillomavirus
LGKRKATPT

SEQ ID NO: 124 - protein, 9 aa, human papillomavirus
CEEASVTVV

SEQ ID NO: 125 - protein, 9 aa, human papillomavirus
LWLPSEATV

SEQ ID NO: 126 - protein, 9 aa, human papillomavirus
IPIVPGSPQ

SEQ ID NO: 127 - protein, 9 aa, human papillomavirus
MADPAAATK

SEQ ID NO: 128 - protein, 9 aa, human papillomavirus
KPYWLQRAQ

SEQ ID NO: 129 - protein, 9 aa, human papillomavirus
DPAGTNGEE

SEQ ID NO: 130 - protein, 9 aa, human papillomavirus
LAKFKELYG

SEQ ID NO: 131 - protein, 9 aa, human papillomavirus
IGNKQTLRT

SEQ ID NO: 132 - protein, 9 aa, human papillomavirus
GNQLFVTVV

SEQ ID NO: 133 - protein, 9 aa, human papillomavirus
DAGDFYLHP

SEQ ID NO: 134 - protein, 9 aa, human papillomavirus
YGNTEVETQ

SEQ ID NO: 135 - protein, 9 aa, human papillomavirus
TPPRPIPKP

SEQ ID NO: 136 - protein, 9 aa, human papillomavirus
AAMLAKFKE

SEQ ID NO: 137 - protein, 9 aa, human papillomavirus
PFDENGNPV

SEQ ID NO: 138 - protein, 9 aa, human papillomavirus
LGIGTGSGT

SEQ ID NO: 139 - protein, 9 aa, human papillomavirus
ATKYPLLKL

SEQ ID NO: 140 - protein, 9 aa, human papillomavirus
GEDLVDFIV

SEQ ID NO: 141 - protein, 9 aa, human papillomavirus
INHQVVPTL

SEQ ID NO: 142 - protein, 9 aa, human papillomavirus
TPSIADSIK

SEQ ID NO: 143 - protein, 9 aa, human papillomavirus
ICEEASVTV

SEQ ID NO: 144 - protein, 9 aa, human papillomavirus
LYLHIQSLA

SEQ ID NO: 145 - protein, 9 aa, human papillomavirus
LADTNSNAS

SEQ ID NO: 146 - protein, 9 aa, human papillomavirus
DYLTQAETE

SEQ ID NO: 147 - protein, 9 aa, human papillomavirus
SLIPIVPGS

SEQ ID NO: 148 - protein, 9 aa, human papillomavirus
RAAKRRLFE

SEQ ID NO: 149 - protein, 9 aa, Artificial Sequence
  Synthetic Sequence
  MISC_FEATURE (1)..(1): X19 is F, W, Y, A, or I
  MISC_FEATURE (2)..(2): X20 is W, Y, F, G, I, L, A, C, S, R, T, Q, or V
  MISC_FEATURE (3)..(3): X21 is L, N, D, W, F, P, Q, V, I, E, R, A, C, T, Y, H, or S
  MISC_FEATURE (4)..(4): X22 is Q, D, P, N, S, A, E, L, F, C, W, H, I, T, G, or K
  MISC_FEATURE (5)..(5): X23 is P, D, or H
  MISC_FEATURE (6)..(6): X24 is L, E, N, F, Y, G, P, T, D, A, Q, I, V, or H
  MISC_FEATURE (7)..(7): X25 is A, W, F, Y, E, Q, V, G, L, P, or M
  MISC_FEATURE (8)..(8): X26 is D, Y, T, A, W, P, F, S, Q, E, K, R, H, V, or C
  MISC_FEATURE (9)..(9): X27 is A, F, L, W, P, H, V, S, N, C, E, or M
XXXXXXXXX

SEQ ID NO: 150 - protein, 9 aa, Lactobacillus floricola
QKQLEILGC

SEQ ID NO: 151 - protein, 9 aa, human papillomavirus
GGQTVQVYF

SEQ ID NO: 152 - protein, 9 aa, human papillomavirus
QATTKDGNS

SEQ ID NO: 153 - protein, 9 aa, human papillomavirus
KNGNPVYEI

SEQ ID NO: 154 - protein, 9 aa, human papillomavirus
HRFSTSDDT

SEQ ID NO: 155 - protein, 9 aa, human papillomavirus
KGGQTVQVY

SEQ ID NO: 156 - protein, 9 aa, human papillomavirus
KSRLTVAKG

SEQ ID NO: 157 - protein, 9 aa, human papillomavirus
ICGHYIILF

SEQ ID NO: 158 - protein, 9 aa, human papillomavirus
QHRFSTSDD

SEQ ID NO: 159 - protein, 9 aa, human papillomavirus
KQGAMLAVF

SEQ ID NO: 160 - protein, 9 aa, human papillomavirus
KAHKAIELQ

SEQ ID NO: 161 - protein, 9 aa, human papillomavirus
SIVDLSTHF

SEQ ID NO: 162 - protein, 9 aa, human papillomavirus
ETLSERLSC

SEQ ID NO: 163 - protein, 9 aa, Artificial Sequence
  Synthetic Sequence
  MISC_FEATURE (1)..(1): X28 is G, W, F, Y, P, R, C, I or L
  MISC_FEATURE (2)..(2): X29 is G, P, D, F, A, Q, Y or S
  MISC_FEATURE (3)..(3): X30 is Q, W, Y, H, R, V, F, L, P, A, D, G or S
  MISC_FEATURE (4)..(4): X31 is T, W, A, F, P, G, H, R, Y, D, N, Q or S
  MISC_FEATURE (5)..(5): X32 is V, W, G or T
  MISC_FEATURE (6)..(6): X33 is Q, N, E, G, P or W
  MISC_FEATURE (7)..(7): X34 is V, D, T or A
  MISC_FEATURE (8)..(8): X35 is Y, D, W or F
  MISC_FEATURE (9)..(9): X36 is F or W
XXXXXXXXX

SEQ ID NO: 164 - protein, 9 aa, human papillomavirus
IGARIHYFY

SEQ ID NO: 165 - protein, 9 aa, human papillomavirus
LLKLLGSTW

SEQ ID NO: 166 - protein, 9 aa, human papillomavirus
QTQLCILGC

SEQ ID NO: 167 - protein, 9 aa, human papillomavirus
TAPTVSACT